(19) United States
(12) Patent Application Publication
(10) Pub. No.: US 2022/0331274 A1
(43) Pub. Date: Oct. 20, 2022

(54) METHODS OF TREATING CANCER

(71) Applicant: Inspirna, Inc., New York, NY (US)

(72) Inventors: Masoud Fakhr TAVAZOIE, New York, NY (US); Robert WASSERMAN, Merion Station, PA (US); David M. DARST, Jr., New York, NY (US)

(21) Appl. No.: 17/641,904

(22) PCT Filed: Sep. 10, 2020
(86) PCT No.: PCT/US2020/050075

§ 371 Date: Mar. 10, 2022

Related U.S. Application Data
(60) Provisional application No. 62/898,872, filed on Sep. 11, 2019.

(51) Int. Cl. A61K 31/195; A61K 31/337; A61K 39/395; A61K 31/519; A61K 31/714; A61K 31/282; A61K 33/243; A61K 31/40; A61K 45/06; A61P 35/00

(52) U.S. Cl. CPC A61K 31/195; A61K 31/337; A61K 39/3955; A61K 31/519; A61K 31/714; A61K 31/282; A61K 33/243; A61K 31/40; A61K 45/06; A61P 35/00

(57) ABSTRACT

The invention features methods of treating cancer with 2-[3-[(3R)-3-[[2-chloro-3-(trifluoromethyl)phenyl]methyl-(2,2-diphenylethyl)amino]butoxy]phenyl]acetic acid. The disclosure also provides methods of treating cancer including combinations of 2-[3-[(3R)-3-[[2-chloro-3-(trifluoromethyl)phenyl]methyl-(2,2-diphenylethyl)amino]butoxy]phenyl]acetic acid and additional anti-cancer therapies.

### BACKGROUND

Liver X receptors (LXRs) LXRα and LXRβ are oxysterol-activated nuclear receptors that regulate expression of genes involved in cholesterol homeostasis and fatty acid metabolism. As such, they have been well characterized in the context of their ability to modulate expression of transporters such as the ABC family of genes that are crucial for cholesterol and triglyceride (TG) metabolism. LXR activation also results in expression of Apolipoprotein E (ApoE), which regulates three tumorigenic features. This includes suppression of cancer cell invasion by binding to low-density lipoprotein receptor-related protein 1 (LRP1) receptors on cancer cells, suppression of angiogenesis by inhibiting endothelial recruitment by binding to LRP8 receptors on endothelial cells, and modulation of the tumoral immune response by regulating the abundance of myeloid derived suppressor cells (MDSCs), a potent immunosuppressive innate immune cell population. Collectively, activation of ApoE expression thus results in inhibition of primary tumor growth and metastatic spread.

As has been demonstrated for melanoma, cancer cells exhibit upregulation of multiple microribonucleic acids (RNAs) that target the ApoE gene and thereby result in the silencing of or reduced expression from the gene. Thus, in animal models increasing expression of ApoE robustly suppresses the growth of melanoma tumors, while genetic knockout of the gene significantly accelerates cancer progression. In fact, the relevance of ApoE in cancer prevention was alluded to in a recent genetic association study that retrospectively analyzed large population sample data from the Framingham Heart Study cohorts. This analysis revealed that carriers of the hypomorphic ApoE4 allele were more likely to die of cancer.

2-[3-[(3R)-3-[[2-chloro-3-(trifluoromethyl)phenyl]methyl-(2,2-diphenylethyl)amino]butoxy]phenyl]acetic acid is a potent small molecule LXR agonist with higher affinity for LXRβ. LXRβ is the predominant isoform expressed in tumor cells of melanoma and a variety of other cancer cell lines. 2-[3-[(3R)-3-[[2-chloro-3-(trifluoromethyl)phenyl]methyl-(2,2-diphenylethyl)amino]butoxy]phenyl]acetic acid exhibits approximately 2.9-fold greater specificity for LXRβ than LXRα with median effective concentration (EC50) values of 118 nM for LXRβ versus 336 nM for LXRα as measured by a cell-based biosensor assay. 2-[3-[(3R)-3-[[2-chloro-3-(trifluoromethyl)phenyl]methyl-(2,2-diphenylethyl)amino]butoxy]phenyl]acetic acid driven transcription of ApoE suppresses invasion of cancer cells through extracellular matrix and recruitment of endothelial cells while also reducing the survival of immune-suppressive MDSCs. LXR agonism has also demonstrated anti-tumor efficacy in syngeneic and human xenograft models of renal cancer. Together, all of these cellular phenomena result in suppression of tumor growth and metastatic spread in animal models.

The efficacy of 2-[3-[(3R)-3-[[2-chloro-3-(trifluoromethyl)phenyl]methyl-(2,2-diphenylethyl)amino]butoxy]phenyl]acetic acid in inhibiting growth of several tumor types has been demonstrated in animal models. In a melanoma xenograft study in non-obese diabetic-severe combined immunodeficiency mice, 2-[3-[(3R)-3-[[2-chloro-3-(trifluoromethyl)phenyl]methyl-(2,2-diphenylethyl)amino]butoxy]phenyl]acetic acid treatment at 25 mg/kg significantly inhibited growth of the xenograft. Similarly, in a syngeneic animal model of mouse melanoma, treatment with 2-[3-[(3R)-3-[[2-chloro-3-(trifluoromethyl)phenyl]methyl-(2,2-diphenylethyl)amino]butoxy]phenyl]acetic acid at a dose of 50 mg/kg in combination with an anti-programmed cell death protein-1 (PD1) therapy to animals bearing tumors that are otherwise resistant to PD-1 inhibition resulted in significant suppression of tumor growth. Furthermore, additive tumor suppressive efficacy of LXR agonism with 3-[3-[N-(2-Chloro-3-trifluoromethylbenzyl)-(2,2-diphenylethyl)amino]propyloxy]phenylacetic acid hydrochloride (GW3965), a close structural analog of 2-[3-[(3R)-3-[[2-chloro-3-(trifluoromethyl)phenyl]methyl-(2,2-diphenylethyl)amino]butoxy]phenyl]acetic acid, at 100 mg/kg has been observed in a syngeneic animal model of melanoma, in combination with anti-cytotoxic T-lymphocyte-associated protein 4 (CTLA-4). Additive anti-tumor efficacy has also been shown in combination with GW3965 and dacarbazine in a syngeneic melanoma animal model and a BRAF wild-type human melanoma xenograft model at 100 mg/kg. Anti-tumor activity of 2-[3-[(3R)-3-[[2-chloro-3-(trifluoromethyl)phenyl]methyl-(2,2-diphenylethyl)amino]butoxy]phenyl]acetic acid has also been demonstrated in animal models of glioblastoma, triple negative breast cancer (TNBC) and lung cancer. In aggregate, these data demonstrate the broad anti-tumor activity of 2-[3-[(3R)-3-[[2-chloro-3-(trifluoromethyl)phenyl]methyl-(2,2-diphenylethyl)amino]butoxy]phenyl]acetic acid and support the advancement of the drug into clinical development for treatment of various advanced cancers. Accordingly, the development of dosing regimens which result in efficacy while minimizing adverse events for the treatment of cancer are needed.

### SUMMARY OF THE INVENTION

The invention features methods of treating cancer by administering 2-[3-[(3R)-3-[[2-chloro-3-(trifluoromethyl)phenyl]methyl-(2,2-diphenylethyl)amino]butoxy]phenyl]acetic acid at least once daily for four to six days followed by one to three days without administration of 2-[3-[(3R)-3-[[2-chloro-3-(trifluoromethyl)phenyl]methyl-(2,2-diphenylethyl)amino]butoxy]phenyl]acetic acid. The inventors have discovered that this dosing schedule surprisingly results in similar efficacy to daily dosing, but a decreased risk of adverse events, e.g., neutropenia.

Accordingly, in one aspect, the invention features a method of increasing the level (e.g., an increase by 5%, 10%, 15%, 20%, 25%, 30%, 35%, 40%, 45%, 50%, 55%, 60%, 65%, 70%, 75%, 80%, 85%, 90%, 95%, 100%, 150%, 200%, 300%, 400%, 500%, or more; or an increase by more than 1.2-fold, 1.4-fold, 1.5-fold, 1.8-fold, 2.0-fold, 3.0-fold, 3.5-fold, 4.5-fold, 5.0-fold, 10-fold, 15-fold, 20-fold, 30-fold, 40-fold, 50-fold, 100-fold, 1000-fold, or more) of ABCA1, ABCG1, ABCG5, ABCG8, SREBP1, ApoE, and/or cholesteryl ester transfer protein mRNA in a subject. This method includes administering to the subject an effective amount of 2-[3-[(3R)-3-[[2-chloro-3-(trifluoromethyl)phenyl]methyl-(2,2-diphenylethyl)amino]butoxy]phenyl]acetic acid, or a pharmaceutically acceptable salt thereof, at least once daily for four to six days (e.g., four days, five days, or six days) followed by one to three days (e.g., one day, two days, or three days) without administration of 2-[3-[(3R)-3-[[2-chloro-3-(trifluoromethyl)phenyl]methyl-(2,2-diphenylethyl)amino]butoxy]phenyl]acetic acid, or a pharmaceutically acceptable salt thereof.

In some embodiments of any of the methods of the invention, the level of ABCA1, ABCG1, ABCG5, ABCG8, SREBP1, ApoE, and/or cholesteryl ester transfer protein mRNA is determined in the tumor microenviroment (e.g., by determining the level in a tumor sample such as a tumor sample from a biopsy). In some embodiments of any of the methods of the invention, the level of ABCA1, ABCG1, ABCG5, ABCG8, SREBP1, ApoE, and/or cholesteryl ester transfer protein mRNA is determined systemically (e.g., by determining the level in a plasma or blood sample).

In an aspect, the invention features a method of decreasing the level (e.g., a decrease by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 100%, about 150%, about 200%, about 300%, about 400%, about 500%, or more; a decrease of more than about 10%, about 15%, about 20%, about 50%, about 75%, about 100%, or about 200%, as compared to a reference) of myeloid derived suppressor cells (e.g., monocytic and/or granulocytic myeloid derived suppressor cells) in a subject. This method includes administering to the subject an effective amount of 2-[3-[(3R)-3-[[2-chloro-3-(trifluoromethyl)phenyl]methyl-(2,2-diphenylethyl)amino]butoxy]phenyl]acetic acid, or a pharmaceutically acceptable salt thereof, at least once daily for four to six days (e.g., four days, five days, or six days) followed by one to three days (e.g., one day, two days, or three days) without administration of 2-[3-[(3R)-3-[[2-chloro-3-(trifluoromethyl)phenyl]methyl-(2,2-diphenylethyl)amino]butoxy]phenyl]acetic acid, or a pharmaceutically acceptable salt thereof.

In some embodiments of any of the methods of the invention, the level of myeloid derived suppressor cells is determined in the tumor microenviroment (e.g., by determining the level in a tumor sample such as a tumor sample from a biopsy). In some embodiments of any of the methods of the invention, the level of myeloid derived suppressor cells is determined systemically (e.g., by determining the level in a plasma or blood sample). In some embodiments of any of the methods of the invention, the myeloid derived suppressor cells are monocytic myeloid derived suppressor cells (e.g., circulating monocytic myeloid derived suppressor cells). In some embodiments of any of the methods of the invention, the myeloid derived suppressor cells are granulocytic myeloid derived suppressor cells. In some embodiments of any of the methods of the invention, the myeloid derived suppressor cells express CD11b(+), Lin(−), HLA-DR(low/−), and/or CD14(+) on their surface. In some embodiments of any of the methods of the invention, the myeloid derived suppressor cells express CD11b(+), Lin(−), HLA-DR(low/−), and CD14(+) on their surface. In some embodiments of any of the methods of the invention, the myeloid derived suppressor cells express CD11 b(+), Lin(−), HLA-DR(low/−), and/or CD15(+) on their surface. In some embodiments of any of the methods of the invention, the myeloid derived suppressor cells express CD11b(+), Lin(−), HLA-DR(low/−), and CD15(+) on their surface. In some embodiments, the myeloid derived suppressor cells express CD33(+), HLA-DR(low/−), and CD15(+). In some embodiments of any of the methods of the invention, the myeloid derived suppressor cells are any known in the art, for example, those described in Talmadge et al. Nat. Rev. Cancer 2013 13(10):739-752.

In an aspect, the invention features a method of increasing the level (e.g., an increase by 5%, 10%, 15%, 20%, 25%, 30%, 35%, 40%, 45%, 50%, 55%, 60%, 65%, 70%, 75%, 80%, 85%, 90%, 95%, 100%, 150%, 200%, 300%, 400%, 500%, or more; or an increase by more than 1.2-fold, 1.4-fold, 1.5-fold, 1.8-fold, 2.0-fold, 3.0-fold, 3.5-fold, 4.5-fold, 5.0-fold, 10-fold, 15-fold, 20-fold, 30-fold, 40-fold, 50-fold, 100-fold, 1000-fold, or more) of activated T-cells in a subject. This method includes administering to the subject an effective amount of 2-[3-[(3R)-3-[[2-chloro-3-(trifluoromethyl)phenyl]methyl-(2,2-diphenylethyl)amino]butoxy]phenyl]acetic acid, or a pharmaceutically acceptable salt thereof, at least once daily for four to six days (e.g., four days, five days, or six days) followed by one to three days (e.g., one day, two days, or three days) without administration of 2-[3-[(3R)-3-[[2-chloro-3-(trifluoromethyl)phenyl]methyl-(2,2-diphenylethyl)amino]butoxy]phenyl]acetic acid, or a pharmaceutically acceptable salt thereof.

In some embodiments, the activated T-cells are PD1+, GITR+, or Lag3+CD8 T-cells. In some embodiments of any of the methods of the invention, the level of activated T-cells is determined in the tumor microenviroment (e.g., by determining the level in a tumor sample such as a tumor sample from a biopsy). In some embodiments of any of the methods of the invention, the level of activated T-cells is determined systemically (e.g., by determining the level in a plasma or blood sample).

In some embodiments of any of the foregoing methods, the level of MDSCs and/or activated T-cells may be determined as described in Iclozan et al. Cancer Immunol. Immunother. 2013, 62(5): 909-918. In some embodiments of any of the foregoing methods, the level of MDSCs and/or activated T-cells may be determined as described in Kitano et al. Cancer Immunol. Res. 2014, 2(8); 812-821.

In an aspect, the invention features a method of increasing (e.g., an increase by 5%, 10%, 15%, 20%, 25%, 30%, 35%, 40%, 45%, 50%, 55%, 60%, 65%, 70%, 75%, 80%, 85%, 90%, 95%, 100%, 150%, 200%, 300%, 400%, 500%, or more; or an increase by more than 1.2-fold, 1.4-fold, 1.5-fold, 1.8-fold, 2.0-fold, 3.0-fold, 3.5-fold, 4.5-fold, 5.0-fold, 10-fold, 15-fold, 20-fold, 30-fold, 40-fold, 50-fold, 100-fold, 1000-fold, or more) ApoE levels in a subject. This method includes administering to the subject an effective amount of 2-[3-[(3R)-3-[[2-chloro-3-(trifluoromethyl)phenyl]methyl-(2,2-diphenylethyl)amino]butoxy]phenyl]acetic acid, or a pharmaceutically acceptable salt thereof, at least once daily for four to six days (e.g., four days, five days, or six days) followed by one to three days (e.g., one day, two days, or three days) without administration of 2-[3-[(3R)-3-[[2-chloro-3-(trifluoromethyl)phenyl]methyl-(2,2-diphenylethyl)amino]butoxy]phenyl]acetic acid, or a pharmaceutically acceptable salt thereof.

In certain embodiments, the LXR agonist increases the expression level of ApoE at least 2.5-fold in vitro. In some embodiments of any of the methods of the invention, the level of ApoE is determined in the tumor microenviroment (e.g., by determining the level in a tumor sample such as a tumor sample from a biopsy). In some embodiments of any of the methods of the invention, the level of ApoE is determined systemically (e.g., by determining the level in a plasma or blood sample).

In an aspect, the invention features a method of treating ApoE-related cancer in a subject in need thereof. This method includes administering to the subject an effective amount of 2-[3-[(3R)-3-[[2-chloro-3-(trifluoromethyl)phenyl]methyl-(2,2-diphenylethyl)amino]butoxy]phenyl]acetic acid, or a pharmaceutically acceptable salt thereof, at least once daily for four to six days (e.g., four days, five days, or six days) followed by one to three days (e.g., one day, two days, or three days) without administration of 2-[3-[(3R)-3-[[2-chloro-3-(trifluoromethyl)phenyl]methyl-(2,2-diphenylethyl)amino]butoxy]phenyl]acetic acid, or a pharmaceutically acceptable salt thereof.

In some embodiments of any of the foregoing methods, the method includes administering to the subject an effective amount of 2-[3-[(3R)-3-[[2-chloro-3-(trifluoromethyl)phenyl]methyl-(2,2-diphenylethyl)amino]butoxy]phenyl]acetic acid, or a pharmaceutically acceptable salt thereof, at least once daily for four days followed by one day without administration of 2-[3-[(3R)-3-[[2-chloro-3-(trifluoromethyl)phenyl]methyl-(2,2-diphenylethyl)amino]butoxy]phenyl]acetic acid, or a pharmaceutically acceptable salt thereof.

In some embodiments of any of the foregoing methods, the method includes administering to the subject an effective amount of 2-[3-[(3R)-3-[[2-chloro-3-(trifluoromethyl)phenyl]methyl-(2,2-diphenylethyl)amino]butoxy]phenyl]acetic acid, or a pharmaceutically acceptable salt thereof, at least once daily for four days followed by two days without administration of 2-[3-[(3R)-3-[[2-chloro-3-(trifluoromethyl)phenyl]methyl-(2,2-diphenylethyl)amino]butoxy]phenyl]acetic acid, or a pharmaceutically acceptable salt thereof.

In some embodiments of any of the foregoing methods, the method includes administering to the subject an effective amount of 2-[3-[(3R)-3-[[2-chloro-3-(trifluoromethyl)phenyl]methyl-(2,2-diphenylethyl)amino]butoxy]phenyl]acetic acid, or a pharmaceutically acceptable salt thereof, at least once daily for four days followed by three days without administration of 2-[3-[(3R)-3-[[2-chloro-3-(trifluoromethyl)phenyl]methyl-(2,2-diphenylethyl)amino]butoxy]phenyl]acetic acid, or a pharmaceutically acceptable salt thereof.

In some embodiments of any of the foregoing methods, the method includes administering to the subject an effective amount of 2-[3-[(3R)-3-[[2-chloro-3-(trifluoromethyl)phenyl]methyl-(2,2-diphenylethyl)amino]butoxy]phenyl]acetic acid, or a pharmaceutically acceptable salt thereof, at least once daily for five days followed by one day without administration of 2-[3-[(3R)-3-[[2-chloro-3-(trifluoromethyl)phenyl]methyl-(2,2-diphenylethyl)amino]butoxy]phenyl]acetic acid, or a pharmaceutically acceptable salt thereof.

In some embodiments of any of the foregoing methods, the method includes administering to the subject an effective amount of 2-[3-[(3R)-3-[[2-chloro-3-(trifluoromethyl)phenyl]methyl-(2,2-diphenylethyl)amino]butoxy]phenyl]acetic acid, or a pharmaceutically acceptable salt thereof, at least once daily for five days followed by two days without administration of 2-[3-[(3R)-3-[[2-chloro-3-(trifluoromethyl)phenyl]methyl-(2,2-diphenylethyl)amino]butoxy]phenyl]acetic acid, or a pharmaceutically acceptable salt thereof.

In some embodiments of any of the foregoing methods, the method includes administering to the subject an effective amount of 2-[3-[(3R)-3-[[2-chloro-3-(trifluoromethyl)phenyl]methyl-(2,2-diphenylethyl)amino]butoxy]phenyl]acetic acid, or a pharmaceutically acceptable salt thereof, at least once daily for five days followed by three days without administration of 2-[3-[(3R)-3-[[2-chloro-3-(trifluoromethyl)phenyl]methyl-(2,2-diphenylethyl)amino]butoxy]phenyl]acetic acid, or a pharmaceutically acceptable salt thereof.

In some embodiments of any of the foregoing methods, the method includes administering to the subject an effective amount of 2-[3-[(3R)-3-[[2-chloro-3-(trifluoromethyl)phenyl]methyl-(2,2-diphenylethyl)amino]butoxy]phenyl]acetic acid, or a pharmaceutically acceptable salt thereof, at least once daily for six days followed by one day without administration of 2-[3-[(3R)-3-[[2-chloro-3-(trifluoromethyl)phenyl]methyl-(2,2-diphenylethyl)amino]butoxy]phenyl]acetic acid, or a pharmaceutically acceptable salt thereof.

In some embodiments of any of the foregoing methods, the method includes administering to the subject an effective amount of 2-[3-[(3R)-3-[[2-chloro-3-(trifluoromethyl)phenyl]methyl-(2,2-diphenylethyl)amino]butoxy]phenyl]acetic acid, or a pharmaceutically acceptable salt thereof, at least once daily for six days followed by two days without administration of 2-[3-[(3R)-3-[[2-chloro-3-(trifluoromethyl)phenyl]methyl-(2,2-diphenylethyl)amino]butoxy]phenyl]acetic acid, or a pharmaceutically acceptable salt thereof.

In some embodiments of any of the foregoing methods, the method includes administering to the subject an effective amount of 2-[3-[(3R)-3-[[2-chloro-3-(trifluoromethyl)phenyl]methyl-(2,2-diphenylethyl)amino]butoxy]phenyl]acetic acid, or a pharmaceutically acceptable salt thereof, at least once daily for six days followed by three days without administration of 2-[3-[(3R)-3-[[2-chloro-3-(trifluoromethyl)phenyl]methyl-(2,2-diphenylethyl)amino]butoxy]phenyl]acetic acid, or a pharmaceutically acceptable salt thereof.

In some embodiments of any of the foregoing methods, the method includes administering the effective amount of 2-[3-[(3R)-3-[[2-chloro-3-(trifluoromethyl)phenyl]methyl-(2,2-diphenylethyl)amino]butoxy]phenyl]acetic acid, or a pharmaceutically acceptable salt thereof, twice daily.

In some embodiments of any of the foregoing methods, the method includes administering the effective amount of 2-[3-[(3R)-3-[[2-chloro-3-(trifluoromethyl)phenyl]methyl-(2,2-diphenylethyl)amino]butoxy]phenyl]acetic acid, or a pharmaceutically acceptable salt thereof, twice daily for five days followed by two days without administration of 2-[3-[(3R)-3-[[2-chloro-3-(trifluoromethyl)phenyl]methyl-(2,2-diphenylethyl)amino]butoxy]phenyl]acetic acid, or a pharmaceutically acceptable salt thereof.

In an aspect, the invention features a method of treating ApoE-related cancer in a subject in need thereof. This method includes administering to the subject about 80 mg of 2-[3-[(3R)-3-[[2-chloro-3-(trifluoromethyl)phenyl]methyl-(2,2-diphenylethyl)amino]butoxy]phenyl]acetic acid, or a pharmaceutically acceptable salt thereof, wherein for each seven day period of treatment, the 2-[3-[(3R)-3-[[2-chloro-3-(trifluoromethyl)phenyl]methyl-(2,2-diphenylethyl)amino]butoxy]phenyl]acetic acid, or a pharmaceutically acceptable salt thereof is administered twice daily for five consecutive days followed by two consecutive days without administration of 2-[3-[(3R)-3-[[2-chloro-3-(trifluoromethyl)phenyl]methyl-(2,2-diphenylethyl)amino]butoxy]phenyl]acetic acid, or a pharmaceutically acceptable salt thereof.

In an aspect, the invention features a method of treating ApoE-related cancer in a subject in need thereof. This method includes administering to the subject about 90 mg of 2-[3-[(3R)-3-[[2-chloro-3-(trifluoromethyl)phenyl]methyl-(2,2-diphenylethyl)amino]butoxy]phenyl]acetic acid, or a pharmaceutically acceptable salt thereof, wherein for each seven day period of treatment, the 2-[3-[(3R)-3-[[2-chloro-3-(trifluoromethyl)phenyl]methyl-(2,2-diphenylethyl)amino]butoxy]phenyl]acetic acid, or a pharmaceutically acceptable salt thereof is administered twice daily for five consecutive days followed by two consecutive days without administration of 2-[3-[(3R)-3-[[2-chloro-3-(trifluoromethyl)phenyl]methyl-(2,2-diphenylethyl)amino]butoxy]phenyl]acetic acid, or a pharmaceutically acceptable salt thereof.

In an aspect, the invention features a method of treating ApoE-related cancer in a subject in need thereof. This method includes administering to the subject about 100 mg of 2-[3-[(3R)-3-[[2-chloro-3-(trifluoromethyl)phenyl]methyl-(2,2-diphenylethyl)amino]butoxy]phenyl]acetic acid, or a pharmaceutically acceptable salt thereof, wherein for each seven day period of treatment, the 2-[3-[(3R)-3-[[2-chloro-3-(trifluoromethyl)phenyl]methyl-(2,2-diphenylethyl)amino]butoxy]phenyl]acetic acid, or a pharmaceutically acceptable salt thereof is administered twice daily for five consecutive days followed by two consecutive days without administration of 2-[3-[(3R)-3-[[2-chloro-3-(trifluoromethyl)phenyl]methyl-(2,2-diphenylethyl)amino]butoxy]phenyl]acetic acid, or a pharmaceutically acceptable salt thereof.

In an aspect, the invention features a method of treating ApoE-related cancer in a subject in need thereof. This method includes administering to the subject about 110 mg of 2-[3-[(3R)-3-[[2-chloro-3-(trifluoromethyl)phenyl]methyl-(2,2-diphenylethyl)amino]butoxy]phenyl]acetic acid, or a pharmaceutically acceptable salt thereof, wherein for each seven day period of treatment, the 2-[3-[(3R)-3-[[2-chloro-3-(trifluoromethyl)phenyl]methyl-(2,2-diphenylethyl)amino]butoxy]phenyl]acetic acid, or a pharmaceutically acceptable salt thereof is administered twice daily for five consecutive days followed by two consecutive days without administration of 2-[3-[(3R)-3-[[2-chloro-3-(trifluoromethyl)phenyl]methyl-(2,2-diphenylethyl)amino]butoxy]phenyl]acetic acid, or a pharmaceutically acceptable salt thereof.

In an aspect, the invention features a method of treating ApoE-related cancer in a subject in need thereof. This method includes administering to the subject about 120 mg of 2-[3-[(3R)-3-[[2-chloro-3-(trifluoromethyl)phenyl]methyl-(2,2-diphenylethyl)amino]butoxy]phenyl]acetic acid, or a pharmaceutically acceptable salt thereof, wherein for each seven day period of treatment, the 2-[3-[(3R)-3-[[2-chloro-3-(trifluoromethyl)phenyl]methyl-(2,2-diphenylethyl)amino]butoxy]phenyl]acetic acid, or a pharmaceutically acceptable salt thereof is administered twice daily for five consecutive days followed by two consecutive days without administration of 2-[3-[(3R)-3-[[2-chloro-3-(trifluoromethyl)phenyl]methyl-(2,2-diphenylethyl)amino]butoxy]phenyl]acetic acid, or a pharmaceutically acceptable salt thereof.

In an aspect, the invention features a method of treating ApoE-related cancer in a subject in need thereof. This method includes administering to the subject about 160 mg of 2-[3-[(3R)-3-[[2-chloro-3-(trifluoromethyl)phenyl]methyl-(2,2-diphenylethyl)amino]butoxy]phenyl]acetic acid, or a pharmaceutically acceptable salt thereof, wherein for each seven day period of treatment, the 2-[3-[(3R)-3-[[2-chloro-3-(trifluoromethyl)phenyl]methyl-(2,2-diphenylethyl)amino]butoxy]phenyl]acetic acid, or a pharmaceutically acceptable salt thereof is administered twice daily for five consecutive days followed by two consecutive days without administration of 2-[3-[(3R)-3-[[2-chloro-3-(trifluoromethyl)phenyl]methyl-(2,2-diphenylethyl)amino]butoxy]phenyl]acetic acid, or a pharmaceutically acceptable salt thereof.

In some embodiments of any of the foregoing methods, the ApoE-related cancer is breast cancer, colon cancer, renal cell cancer, lung cancer, hepatocellular carcinoma, gastric cancer, ovarian cancer, pancreatic cancer, esophageal cancer, prostate cancer, sarcoma, bladder cancer, neuroendocrine cancer, lymphoma, squamous cell carcinoma of the head and neck, or melanoma.

In some embodiments of any of the foregoing methods, the ApoE-related cancer is breast cancer such as triple negative breast cancer, colon cancer, renal cell cancer, non-small cell lung cancer (e.g., non-squamous non-small cell lung cancer), hepatocellular carcinoma, gastric cancer, ovarian cancer, pancreatic cancer, esophageal cancer, prostate cancer, sarcoma, glioblastoma, diffuse large B-cell lymphoma, leukemia (e.g., acute myeloid leukemia), or melanoma. In some embodiments of any of the foregoing methods, the ApoE-related cancer is melanoma. In some embodiments of any of the foregoing methods, the ApoE-related cancer is breast cancer. In some embodiments of any of the foregoing methods, the ApoE-related cancer is renal cell cancer. In some embodiments of any of the foregoing methods, the ApoE-related cancer is pancreatic cancer. In some embodiments of any of the foregoing methods, the ApoE-related cancer is non-small cell lung cancer (e.g., non-squamous non-small cell lung cancer). In some embodiments of any of the foregoing methods, the ApoE-related cancer is colon cancer. In some embodiments of any of the foregoing methods, the ApoE-related cancer is ovarian cancer. In some embodiments of any of the foregoing methods, the ApoE-related cancer is glioblastoma. In some embodiments, the ApoE-related cancer is breast cancer. In some embodiments, the ApoE-related cancer is prostate cancer. In some embodiments, the ApoE-related cancer is diffuse large B-cell lymphoma. In some embodiments, the ApoE-related cancer is leukemia (e.g., acute myeloid leukemia). In some embodiments, the ApoE-related cancer is neuroendocrine cancer (e.g., high grade neuroendocrine cancer). In some embodiments, the ApoE-related cancer is small-cell lung cancer. In some embodiments, the ApoE-related cancer is squamous cell carcinoma of the head and neck.

In particular embodiments, the ApoE-related cancer is melanoma (e.g., metastatic melanoma) that is resistant to, or has failed to respond to prior treatment with, vemurafenib, dacarbazine, interferon therapy, a CTLA-4 inhibitor, a BRAF inhibitor, a MEK inhibitor, a PD1 inhibitor, a PDL-1 inhibitor, and/or a CAR-T therapy. In some embodiments, the ApoE-related cancer is glioblastoma that is resistant to, or has failed to respond to prior treatment with, temozolimide, radiotherapy, avastin, irinotecan, a VEGFR2 inhibitor, a CAR-T therapy, and/or an mTOR inhibitor. In some embodiments, the ApoE-related cancer is non-small cell lung cancer such as metastatic non-small cell lung cancer (e.g., EGFR-wild type non-small cell lung cancer, squamous non-small cell lung cancer, or non-squamous non-small cell lung cancer) that is resistant to, or has failed to respond to prior treatment with, an EGFR inhibitor, platinum agents (e.g., carboplatin), avastin, an ALK inhibitor, a MET inhibitor, a taxane (e.g., paclitaxel and/or doceltaxel), gemzar, alimta, radiotherapy, a PD1 inhibitor, a PDL1 inhibitor, and/or a CAR-T therapy. In some embodiments, the ApoE-related cancer is a breast cancer (e.g., triple negative breast cancer) that is resistant to, or has failed to respond to prior treatment with, herceptin, perjeta, tamoxifen, xeloda, docetaxel, carboplatin, paclitaxel, abraxane, adriamycin, gemcitabine, avastin, halaven, neratinib, a PARP inhibitor, a PD1 inhibitor, a PDL1 inhibitor, a CAR-T therapy, ARN810, and/or an mTOR inhibitor. In some embodiments, the ApoE-related cancer is ovarian cancer (e.g., metastatic ovarian cancer) that is resistant to, or has failed to respond to prior treatment with, a PARP inhibitor, avastin, platinum agents such as carboplatin, paclitaxel, docetaxel, topotecan, gemzar, a VEGR2 inhibitor, a folate receptor antagonist, a PD1 inhibitor, a PDL1 inhibitor, a CAR-T therapy, demcizumab, and/or fosbretabulin.

In some embodiments of any of the foregoing methods, the effective amount of 2-[3-[(3R)-3-[[2-chloro-3-(trifluoromethyl)phenyl]methyl-(2,2-diphenylethyl)amino]butoxy]phenyl]acetic acid, or a pharmaceutically acceptable salt thereof is about 80 mg to about 160 mg (e.g., about 80 mg, about 90 mg, about 100 mg, about 110 mg, about 120 mg, about 80 mg to 100 mg, about 90 mg to 110 mg, about 100 mg to 120 mg, about 90 to 120 mg, or about 110 to 160 mg) per administration. In some embodiments of any of the foregoing methods, the method includes administering 80 mg of 2-[3-[(3R)-3-[[2-chloro-3-(trifluoromethyl)phenyl]methyl-(2,2-diphenylethyl)amino]butoxy]phenyl]acetic acid, or a pharmaceutically acceptable salt thereof, twice daily for five days followed by two days without administration of 2-[3-[(3R)-3-[[2-chloro-3-(trifluoromethyl)phenyl]methyl-(2,2-diphenylethyl)amino]butoxy]phenyl]acetic acid, or a pharmaceutically acceptable salt thereof.

In some embodiments of any of the foregoing methods, the subject has previously received an anti-cancer therapy (e.g., surgery, radiation, chemotherapy, and/or immunotherapy).

In some embodiments of any of the foregoing methods, the method further includes administering an additional anti-cancer therapy (e.g., surgery, radiation, chemotherapy, and/or immunotherapy) to the subject.

In particular embodiments, the antiproliferative is: a chemotherapeutic or cytotoxic agent, a differentiation-inducing agent (e.g. retinoic acid, vitamin D, cytokines), a hormonal agent, an immunological agent, or an anti-angiogenic agent. Chemotherapeutic and cytotoxic agents include, but are not limited to, alkylating agents, cytotoxic antibiotics, antimetabolites, vinca alkaloids, etoposides, and others (e.g., paclitaxel, taxol, docetaxel, taxotere, cis-platinum). A list of additional compounds having antiproliferative activity can be found in L. Brunton, B. Chabner and B. Knollman (eds). Goodman and Gilman's The Pharmacological Basis of Therapeutics, Twelfth Edition, 2011, McGraw Hill Companies, New York, N.Y.

In certain embodiments, the antiproliferative is a PD1 inhibitor, a VEGF inhibitor, a VEGFR2 inhibitor, a PDL1 inhibitor, a BRAF inhibitor, a CTLA-4 inhibitor, a MEK inhibitor, an ERK inhibitor, vemurafenib, dacarbazine, trametinib, dabrafenib, MEDI-4736, an mTOR inhibitor, a CAR-T therapy, abiraterone, enzalutamine, ARN-509, 5-FU, FOLFOX, FOLFIRI, herceptin, xeloda, a PD1 antibody (e.g., pembrolizumab or nivolumab), a PDL-1 antibody, a CTLA-4 antibody (e.g, ipilimumab), ramucirumab, rindopepimut, glembatumumab, vedotin, ANG1005, and/or ANG4043.

In some embodiments of any of the foregoing methods, the additional anti-cancer therapy is an immunotherapy. In some embodiments of any of the foregoing methods, the immunotherapy, when present, is a CTLA-4 inhibitor, a PD1 inhibitor, a PDL1 inhibitor, or adoptive T-cell transfer therapy. In some embodiments, the immunotherapy is a PD-1 inhibitor such as a PD-1 antibody, a PD-L1 inhibitor such as a PD-L1 antibody, a CTLA-4 inhibitor such as a CTLA-4 antibody, a CSF-1R inhibitor, an IDO inhibitor, an A1 adenosine inhibitor, an A2A adenosine inhibitor, an A2B adenosine inhibitor, an A3A adenosine inhibitor, an arginase inhibitor, or an HDAC inhibitor. In some embodiments, the immunotherapy is a PD-1 inhibitor (e.g., nivolumab, pembrolizumab, pidilizumab, BMS 936559, and MPDL3280A). In some embodiments, the immunotherapy is a PD-L1 inhibitor (e.g., atezolizumab and MEDI4736). In some embodiments, the immunotherapy is a PD1 or PDL-1 inhibitor (e.g., toripalimab, sintilimab, camrelizumab, CS1001, tislelizumab, HLX-10, TQB2450, SHR-1316, AK105, GLS-010, AK103, KN035, GB226, KL-A167, or KN046). In some embodiments, the immunotherapy is a PD-L1/TGF-beta trap (e.g., bintrafusp alfa (M7824) or fusion proteins as described in International Patent Publication No. WO2018/205985) In some embodiments, the immunotherapy is a CTLA-4 inhibitor (e.g., ipilimumab). In some embodiments, the immunotherapy is a CSF-1R inhibitor (e.g., pexidartinib and AZD6495). In some embodiments, the immunotherapy is an IDO inhibitor (e.g., norharmane, rosmarinic acid, and alpha-methyl-tryptophan). In some embodiments, the immunotherapy is an A1 adenosine inhibitor (e.g., 8-cyclopentyl-1,3-dimethylxanthine, 8-cyclopentyl-1,3-dipropylxanthine, 8-phenyl-1,3-dipropylxanthine, bamifylline, BG-9719, BG-9928, FK-453, FK-838, rolofylline, or N-0861). In some embodiments, the immunotherapy is an A2A adenosine inhibitor (e.g., ATL-4444, istradefylline, MSX-3, preladenant, SCH-58261, SCH-412,348, SCH-442,416, ST-1535, VER-6623, VER-6947, VER-7835, viadenant, or ZM-241,385). In some embodiments, the immunotherapy is an A2B adenosine inhibitor (e.g., ATL-801, CVT-6883, MRS-1706, MRS-1754, OSIP-339,391, PSB-603, PSB-0788, or PSB-1115). In some embodiments, the immunotherapy is an A3A adenosine inhibitor (e.g., KF-26777, MRS-545, MRS-1191, MRS-1220, MRS-1334, MRS-1523, MRS-3777, MRE-3005-F20, MRE-3008-F20, PSB-11, OT-7999, VUF-5574, and SSR161421). In some embodiments, the immunotherapy is an arginase inhibitor (e.g., an arginase antibody, (2s)-(+)-amino-5-iodoacetamidopentanoic acid, NG-hydroxy-L-arginine, (2S)-(+)-amino-6-iodoacetamidohexanoic acid, or (R)-2-amino-6-borono-2-(2-(piperidin-1-yl)ethyl)hexanoic acid. In some embodiments, the immunotherapy is an HDAC inhibitor (e.g., valproic acid, SAHA, or romidepsin).

In some embodiments, the cancer is a renal cell carcinoma and the antiproliferative is a PD1 inhibitor, a PDL-1 inhibitor, or an mTOR inhibitor. In other embodiments, the cancer is diffuse large B-cell lymphoma and the antiproliferative is a CAR-T therapy. In certain embodiments, the cancer is prostate cancer and the antiproliferative is abiraterone, enzalutamide, or ARN-509. In some embodiments, the cancer is hepatocellular carcinoma, gastric cancer, or esophageal cancer and the antiproliferative is 5-FU, FOLFOX, FOLFIRI, herceptin, or xeloda. In some embodiments, the cancer is sarcoma and the antiproliferative is gemcitabine. In other embodiments, the cancer is pancreatic cancer and the antiproliferative is irinotecan, cisplatin, abraxane, a taxane (e.g., paclitaxel or docetaxel), or capecitabine.

The method may further include administering an antiproliferative selected from the group consisting of alkylating agents, platinum agents, antimetabolites, topoisomerase inhibitors, antitumor antibiotics, antimitotic agents, aromatase inhibitors, thymidylate synthase inhibitors, DNA antagonists, farnesyltransferase inhibitors, pump inhibitors, histone acetyltransferase inhibitors, metalloproteinase inhibitors, ribonucleoside reductase inhibitors, TNF alpha agonists/antagonists, endothelin A receptor antagonist, retinoic acid receptor agonists, immuno-modulators, hormonal and antihormonal agents, photodynamic agents, tyrosine kinase inhibitors, antisense compounds, corticosteroids, HSP90 inhibitors, proteosome inhibitors (for example, NPI-0052), CD40 inhibitors, anti-CSI antibodies, FGFR3 inhibitors, VEGF inhibitors, MEK inhibitors, cyclin D1 inhibitors, NF-kB inhibitors, anthracyclines, histone deacetylases, kinesin inhibitors, phosphatase inhibitors, COX2 inhibitors, mTOR inhibitors, calcineurin antagonists, IMiDs, or other agents used to treat proliferative diseases.

In some embodiments of any of the foregoing methods, the additional anti-cancer therapy includes chemotherapy.

In some embodiments of any of the foregoing methods, the chemotherapy includes docetaxel. In some embodiments, the method includes administering an effective amount of docetaxel to the subject once every seven days. In some embodiments, the effective amount of docetaxel is at least 28 mg/m². In some embodiments, the effective amount of docetaxel is about 28 mg/m² to about 35 mg/m².

In some embodiments of any of the foregoing methods, the additional anti-cancer therapy includes chemotherapy and immunotherapy. In some embodiments of any of the foregoing methods, the anti-cancer therapy includes carboplatin or cisplatin, pemetrexed, and pembrolizumab. In some embodiments of any of the foregoing methods, the method includes administering to the subject an effective amount of pembrolizumab once every twenty-one days. In some embodiments of any of the foregoing methods, the effective amount of pembrolizumab is about 200 mg. In some embodiments of any of the foregoing methods, the method includes administering to the subject an effective amount of carboplatin or cisplatin once every twenty-one days. In some embodiments of any of the foregoing methods, the effective amount of carboplatin or cisplatin is calculated using the formula: Total dose (mg)=(Target area under the curve)×(subject's glomerular filtration rate+25), wherein the target area under the curve is 4 mg/mL*min to 6 mg/mL*min and the subject's glomerular filtration rate was measured by Cr-EDTA clearance. In some embodiments of any of the foregoing methods, the effective amount of carboplatin or cisplatin is about 300 mg/m² to about 360 mg/m². In some embodiments of any of the foregoing methods, the method includes administering to the subject an effective amount of pemetrexed once every twenty-one days. In some embodiments of any of the foregoing methods, the effective amount of pemetrexed is 500 mg/m². In some embodiments of any of the foregoing methods, the method further includes administering to the subject an effective amount of folic acid, vitamin B12, and/or corticosteroids. In some embodiments of any of the foregoing methods, the method includes administering to the subject an effective amount of corticosteroids twice per day for three days prior to administration of pemetrexed.

In some embodiments of any of the foregoing methods, the method further includes administering to the subject an effective amount of a statin (e.g., rosuvastatin or atorvastatin).

In some embodiments of any of the foregoing methods, the method further includes administering to the subject an effective amount of an anti-emetic agent (e.g., ondansetron, granisetron, palonosetron, metoclopramide, haloperidol, dexamethasone, aprepitant, fosaprepitant, lorazepam, dronabinol, prochlorperazine, or chlorpromazine), an anti-diarrheal agent (e.g., an opiate agonist or octreotide), an appetite stimulant (e.g., megestrol acetate, metoclopramide, dronabinol, prednisone, or dexamethasone), a general stimulant, a bisphosphonate (e.g., etidronate, clodronate, tiludronate, pamidronate, neridronate, opladronate, alendronate, ibandronate, risedronate, or zoledronate), a gonadotrophin releasing hormone agonist (e.g., buserelin, histrelin, leuprorelin, triptorelin, goserelin, or nafarelin), and/or growth factors (e.g., filgrastim).

In some embodiments of any of the foregoing methods, the cancer is resistant to an anti-cancer therapy (e.g., platinum-containing chemotherapy, a PD-1 inhibitor, a PD-L1 inhibitor, a CTLA-4 inhibitor, an antimitotic agent, a topoisomerase inhibitor, an antimetabolite, an angiogenesis inhibitor, a kinase inhibitor, and/or an alkylating agent). In some embodiments of any of the foregoing methods, the cancer progressed on or after treatment with an anti-cancer therapy (e.g., platinum-containing chemotherapy, a PD-1 inhibitor, a PD-L1 inhibitor, an angiogenesis inhibitor, a kinase inhibitor, and/or an alkylating agent). In some embodiments of any of the foregoing methods, the cancer has been determined to be, or is predicted to be, resistant to an anti-cancer therapy (e.g., a PD-1 inhibitor, a PD-L1 inhibitor, a CTLA-4 inhibitor, a topoisomerase inhibitor, an antimetabolite, an angiogenesis inhibitor, a kinase inhibitor, and/or an alkylating agent).

In some embodiments of any of the foregoing methods, the cancer has a PDL-1 expression level of less than 1% when tested in an immunohistochemistry assay (e.g., an immunohistochemistry assay with a tumor proportion score). In some embodiments, the cancer has a PDL-1 expression level of about 1% when tested in an immunohistochemistry assay (e.g., an immunohistochemistry assay with a tumor proportion score). In some embodiments, the cancer has a PDL-1 expression level of about 1% to about 49% (e.g., about 1% to about 20%, about 5% to about 30%, about 15% to about 40%, about 25% to about 49%) when tested in an immunohistochemistry assay (e.g., an immunohistochemistry assay with a tumor proportion score). In some embodiments of any of the foregoing methods, the cancer is metastatic and/or locally advanced. In some embodiments of any of the foregoing methods, the cancer is unresectable.

In some embodiments of any of the foregoing methods, the risk of adverse events (e.g., hypertriglyceridemia, hypercholesterolemia, neutropenia, or immune-related adverse events such as interstitial pneumonia, colitis, hypothyroidism, liver dysfunction, skin rash, vitiligo, hypophysitis, type 1 diabetes, renal dysfunction, myasthenia gravis, neuropathy, myositis, and uveitis) is reduced in comparison to administration of the effective amount of 2-[3-[(3R)-3-[[2-chloro-3-(trifluoromethyl)phenyl]methyl-(2,2-diphenylethyl)amino]butoxy]phenyl]acetic acid, or a pharmaceutically acceptable salt thereof, at least once daily for seven consecutive days. In some embodiments of any of the foregoing methods, the risk of neutropenia is reduced in comparison to administration of the effective amount of 2-[3-[(3R)-3-[[2-chloro-3-(trifluoromethyl)phenyl]methyl-(2,2-diphenylethyl)amino]butoxy]phenyl]acetic acid, or a pharmaceutically acceptable salt thereof, at least once daily for seven consecutive days. In some embodiments of any of the foregoing methods, the risk of hypertriglyceridemia and/or hypercholesterolemia is reduced in comparison to administration of the effective amount of 2-[3-[(3R)-3-[[2-chloro-3-(trifluoromethyl)phenyl]methyl-(2,2-diphenylethyl)amino]butoxy]phenyl]acetic acid, or a pharmaceutically acceptable salt thereof, at least once daily for seven consecutive days. In some embodiments of any of the foregoing methods, the risk of immune-related adverse events (e.g., interstitial pneumonia, colitis, hypothyroidism, liver dysfunction, skin rash, vitiligo, hypophysitis, type 1 diabetes, renal dysfunction, myasthenia gravis, neuropathy, myositis, and/or uveitis) is reduced in comparison to administration of the effective amount of 2-[3-[(3R)-3-[[2-chloro-3-(trifluoromethyl)phenyl]methyl-(2,2-diphenylethyl)amino]butoxy]phenyl]acetic acid, or a pharmaceutically acceptable salt thereof, at least once daily for seven consecutive days. In some embodiments, the risk of adverse events may be determined by comparing a group of subjects administered the effective amount of 2-[3-[(3R)-3-[[2-chloro-3-(trifluoromethyl)phenyl]methyl-(2,2-diphenylethyl)amino]butoxy]phenyl]acetic acid, or a pharmaceutically acceptable salt thereof, at least once daily for seven consecutive days and a group of subjects administered the effective amount of 2-[3-[(3R)-3-[[2-chloro-3-(trifluoromethyl)phenyl]methyl-(2,2-diphenylethyl)amino]butoxy]phenyl]acetic acid, or a pharmaceutically acceptable salt thereof, at least once daily for four to six consecutive days followed by one to three days without administration.

In some embodiments of any of the foregoing methods, the level of neutrophils in a sample from the subject are increased in comparison to a mean of the level of neutrophils in a plurality of subjects administered the effective amount of 2-[3-[(3R)-3-[[2-chloro-3-(trifluoromethyl)phenyl]methyl-(2,2-diphenylethyl)amino]butoxy]phenyl]acetic acid, or a pharmaceutically acceptable salt thereof, at least once daily for seven consecutive days. In some embodiments of any of the foregoing methods, the level of triglycerides and/or cholesterol in a sample from the subject are decreased in comparison to a mean of the level of triglycerides and/or cholesterol in a plurality of subjects administered the effective amount of 2-[3-[(3R)-3-[[2-chloro-3-(trifluoromethyl)phenyl] methyl-(2,2-diphenylethyl)amino] butoxy]phenyl]acetic acid, or a pharmaceutically acceptable salt thereof, at least once daily for seven consecutive days.

In some embodiments of any of the foregoing methods, the 2-[3-[(3R)-3-[[2-chloro-3-(trifluoromethyl)phenyl]methyl-(2,2-diphenylethyl)amino]butoxy]phenyl]acetic acid is administered orally.

In some embodiments of any of the foregoing methods, the method includes at least 21 days of treatment (e.g., at least 28 days, at least 3 months, at least 6 months, at least one year).

### Definitions

As used herein, the term “about” represents a value that is in the range of ±10% of the value that follows the term “about.”

As used herein, the term “administration” refers to the administration of a composition (e.g., a compound or a preparation that includes a compound as described herein) to a subject or system. Administration to an animal subject (e.g., to a human) may be by any appropriate route. For example, in some embodiments, administration may be bronchial (including by bronchial instillation), buccal, enteral, interdermal, intra-arterial, intradermal, intragastric, intramedullary, intramuscular, intranasal, intraperitoneal, intrathecal, intravenous, intraventricular, mucosal, nasal, oral, rectal, subcutaneous, sublingual, topical, tracheal (including by intratracheal instillation), transdermal, vaginal, and vitreal.

By “biological sample” or “sample” is meant a fluid or solid sample from a subject. Biological samples may include cells; nucleic acid, protein, or membrane extracts of cells; or blood or biological fluids including (e.g., plasma, serum, saliva, urine, bile). Solid biological samples include samples taken from feces, the rectum, central nervous system, bone, breast tissue, renal tissue, the uterine cervix, the endometrium, the head or neck, the gallbladder, parotid tissue, the prostate, the brain, the pituitary gland, kidney tissue, muscle, the esophagus, the stomach, the small intestine, the colon, the liver, the spleen, the pancreas, thyroid tissue, heart tissue, lung tissue, the bladder, adipose tissue, lymph node tissue, the uterus, ovarian tissue, adrenal tissue, testis tissue, the tonsils, and the thymus. Fluid biological samples include samples taken from the blood, serum, plasma, pancreatic fluid, CSF, semen, prostate fluid, seminal fluid, urine, saliva, sputum, mucus, bone marrow, lymph, and tears. Samples may be obtained by standard methods including, e.g., venous puncture and surgical biopsy. In certain embodiments, the biological sample is a blood, plasma, or serum sample. In some embodiments, the biological sample is a tumor sample from a biopsy.

The term “cancer” refers to any cancer caused by the proliferation of malignant neoplastic cells, such as tumors, neoplasms, carcinomas, sarcomas, leukemias, and lymphomas.

“Cell migration” as used in this application involves the invasion by the cancer cells into the surrounding tissue and the crossing of the vessel wall to exit the vasculature in distal organs of the cancer cell.

By “cell migration cancers” is meant cancers that migrate by invasion by the cancer cells into the surrounding tissue and the crossing of the vessel wall to exit the vasculature in distal organs of the cancer cell.

By “determining the level of a cell type” is meant the detection of a cell type by methods known in the art either directly or indirectly. “Directly determining” means performing a process (e.g., performing an assay or test on a sample or “analyzing a sample” as that term is defined herein) to obtain the physical entity or value. “Indirectly determining” refers to receiving the physical entity or value from another party or source (e.g., a third-party laboratory that directly acquired the physical entity or value). Methods to measure cell levels generally include, but are not limited to, flow cytometry and immunohistochemistry. Exemplary methods are provided herein. In some embodiments of any of the foregoing methods, the level of MDSCs and/or activated T-cells may be determined as described in Iclozan et al. Cancer Immunol. Immunother. 2013, 62(5): 909-918. In some embodiments of any of the foregoing methods, the level of MDSCs and/or activated T-cells may be determined as described in Kitano et al. Cancer Immunol. Res. 2014, 2(8); 812-821.

By “determining the level of a protein or mRNA” is meant the detection of a protein or mRNA by methods known in the art either directly or indirectly. “Directly determining” means performing a process (e.g., performing an assay or test on a sample or “analyzing a sample” as that term is defined herein) to obtain the physical entity or value. “Indirectly determining” refers to receiving the physical entity or value from another party or source (e.g., a third-party laboratory that directly acquired the physical entity or value). Methods to measure protein level generally include, but are not limited to, western blotting, immunoblotting, enzyme-linked immunosorbent assay (ELISA), radioimmunoassay (RIA), immunoprecipitation, immunofluorescence, surface plasmon resonance, chemiluminescence, fluorescent polarization, phosphorescence, immunohistochemical analysis, matrix-assisted laser desorption/ionization time-of-flight (MALDI-TOF) mass spectrometry, liquid chromatography (LC)-mass spectrometry, microcytometry, microscopy, fluorescence activated cell sorting (FACS), and flow cytometry, as well as assays based on a property of a protein including, but not limited to, enzymatic activity or interaction with other protein partners. Methods to measure mRNA and glycan levels are known in the art.

A cancer “determined to be drug resistant,” as used herein, refers to a cancer that is drug resistant, based on unresponsiveness or decreased responsiveness to a chemotherapeutic agent, or is predicted to be drug resistant based on a prognostic assay (e.g., a gene expression assay).

By a “drug resistant” cancer is meant a cancer that does not respond, or exhibits a decreased response to, one or more chemotherapeutic agents (e.g., any agent described herein).

The term “effective amount” means an amount that is sufficient, when administered to a population suffering from or susceptible to a disease, disorder, and/or condition in accordance with a therapeutic dosing regimen, to treat the disease, disorder, and/or condition. In some embodiments, a therapeutically effective amount is one that reduces the incidence and/or severity of, and/or delays onset of, one or more symptoms of the disease, disorder, and/or condition. Those of ordinary skill in the art will appreciate that the term “effective amount” does not in fact require successful treatment be achieved in a particular individual. Rather, an effective amount may be that amount that provides a particular desired pharmacological response in a significant number of subjects when administered to subjects in need of such treatment. It is specifically understood that particular subjects may, in fact, be “refractory” to an “effective amount.” To give but one example, a refractory subject may have a low bioavailability such that clinical efficacy is not obtainable. In some embodiments, reference to an effective amount may be a reference to an amount as measured in one or more specific tissues (e.g., a tissue affected by the disease, disorder or condition) or fluids (e.g., blood, saliva, serum, sweat, tears, urine). Those of ordinary skill in the art will appreciate that, in some embodiments, an effective amount may be formulated and/or administered in a single dose. In some embodiments, an effective amount may be formulated and/or administered in a plurality of doses, for example, as part of a dosing regimen.

As used herein, the term “failed to respond to a prior therapy” or “refractory to a prior therapy,” refers to a cancer that progressed despite treatment with the therapy.

By “level” is meant a level of a cell type, as compared to a reference. The reference can be any useful reference, as defined herein. By a “decreased level” or an “increased level” of a cell type is meant a decrease or increase in cell level, as compared to a reference (e.g., a decrease or an increase by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 100%, about 150%, about 200%, about 300%, about 400%, about 500%, or more; a decrease or an increase of more than about 10%, about 15%, about 20%, about 50%, about 75%, about 100%, or about 200%, as compared to a reference; a decrease or an increase by less than about 0.01-fold, about 0.02-fold, about 0.1-fold, about 0.3-fold, about 0.5-fold, about 0.8-fold, or less; or an increase by more than about 1.2-fold, about 1.4-fold, about 1.5-fold, about 1.8-fold, about 2.0-fold, about 3.0-fold, about 3.5-fold, about 4.5-fold, about 5.0-fold, about 10-fold, about 15-fold, about 20-fold, about 30-fold, about 40-fold, about 50-fold, about 100-fold, about 1000-fold, or more). A level of a cell type may be expressed in mass/vol (e.g., g/dL, mg/mL, μg/mL, ng/mL) or percentage relative to total cells in a sample. In some embodiments of any of the foregoing methods, the reference is a sample from a healthy subject such as a subject that does not have cancer. In some embodiments of any of the foregoing methods, the reference is an artificial sample with a level (e.g., a level of MDSCs such as monocytic and/or granulocytic MDSCs or activated T-cells) shown beneficial in the treatment of a disorder.

As used herein, “metastatic nodule” refers to an aggregation of tumor cells in the body at a site other than the site of the original tumor.

As used herein, “metastatic tumor” refers to a tumor or cancer in which the cancer cells forming the tumor have a high potential to or have begun to, metastasize, or spread from one location to another location or locations within a subject, via the lymphatic system or via hematogenous spread, for example, creating secondary tumors within the subject. Such metastatic behavior may be indicative of malignant tumors. In some cases, metastatic behavior may be associated with an increase in cell migration and/or invasion behavior of the tumor cells.

Examples of cancers that can be defined as metastatic include but are not limited to non-small cell lung cancer (e.g., non-squamous non-small cell lung cancer), breast cancer, ovarian cancer, colorectal cancer, biliary tract cancer, bladder cancer, brain cancer including glioblastomas and medulloblastomas, cervical cancer, choriocarcinoma, endometrial cancer, esophageal cancer, gastric cancer, hematological neoplasms, multiple myeloma, leukemia, intraepithelial neoplasms, liver cancer, lymphomas, neuroblastomas, oral cancer, pancreatic cancer, prostate cancer, sarcoma, skin cancer including melanoma, basocellular cancer, squamous cell cancer, testicular cancer, stromal tumors, germ cell tumors, thyroid cancer, and renal cancer.

As used herein, “migrating cancer” refers to a cancer in which the cancer cells forming the tumor migrate and subsequently grow as malignant implants at a site other than the site of the original tumor. The cancer cells migrate via seeding the surface of the peritoneal, pleural, pericardial, or subarachnoid spaces to spread into the body cavities; via invasion of the lymphatic system through invasion of lymphatic cells and transport to regional and distant lymph nodes and then to other parts of the body; via hematogenous spread through invasion of blood cells; or via invasion of the surrounding tissue. Migrating cancers include metastatic tumors and cell migration cancers, such as ovarian cancer, mesothelioma, and primary lung cancer, each of which is characterized by cellular migration.

“Non-metastatic cell migration cancer” as used herein refers to cancers that do not migrate via the lymphatic system or via haematogenous spread.

As used herein, the term “pharmaceutical composition” refers to an active compound, formulated together with one or more pharmaceutically acceptable carriers. In some embodiments, active compound is present in unit dose amount appropriate for administration in a therapeutic regimen that shows a statistically significant probability of achieving a predetermined therapeutic effect when administered to a relevant population. In some embodiments, pharmaceutical compositions may be specially formulated for administration in solid or liquid form, including those adapted for the following: oral administration, for example, drenches (aqueous or non-aqueous solutions or suspensions), tablets, e.g., those targeted for buccal, sublingual, and systemic absorption, boluses, powders, granules, pastes for application to the tongue; parenteral administration, for example, by subcutaneous, intramuscular, intravenous or epidural injection as, for example, a sterile solution or suspension, or sustained-release formulation; topical application, for example, as a cream, ointment, or a controlled-release patch or spray applied to the skin, lungs, or oral cavity; intravaginally or intrarectally, for example, as a pessary, cream, or foam; sublingually; ocularly; transdermally; or nasally, pulmonary, and to other mucosal surfaces.

A “pharmaceutically acceptable excipient,” as used herein, refers any inactive ingredient (for example, a vehicle capable of suspending or dissolving the active compound) having the properties of being nontoxic and non-inflammatory in a subject. Typical excipients include, for example: antiadherents, antioxidants, binders, coatings, compression aids, disintegrants, dyes (colors), emollients, emulsifiers, fillers (diluents), film formers or coatings, flavors, fragrances, glidants (flow enhancers), lubricants, preservatives, printing inks, sorbents, suspending or dispersing agents, sweeteners, or waters of hydration. Excipients include, but are not limited to: butylated hydroxytoluene (BHT), calcium carbonate, calcium phosphate (dibasic), calcium stearate, croscarmellose, crosslinked polyvinyl pyrrolidone, citric acid, crospovidone, cysteine, ethylcellulose, gelatin, hydroxypropyl cellulose, hydroxypropyl methylcellulose, lactose, magnesium stearate, maltitol, mannitol, methionine, methylcellulose, methyl paraben, microcrystalline cellulose, polyethylene glycol, polyvinyl pyrrolidone, povidone, pregelatinized starch, propyl paraben, retinyl palmitate, shellac, silicon dioxide, sodium carboxymethyl cellulose, sodium citrate, sodium starch glycolate, sorbitol, starch (corn), stearic acid, stearic acid, sucrose, talc, titanium dioxide, vitamin A, vitamin E, vitamin C, and xylitol. Those of ordinary skill in the art are familiar with a variety of agents and materials useful as excipients.

The term “pharmaceutically acceptable salt,” as use herein, refers to those salts of the compounds described here that are, within the scope of sound medical judgment, suitable for use in contact with the tissues of humans and animals without undue toxicity, irritation, allergic response and the like and are commensurate with a reasonable benefit/risk ratio. Pharmaceutically acceptable salts are well known in the art. For example, pharmaceutically acceptable salts are described in: Berge et al., J. Pharmaceutical Sciences 66:1-19, 1977 and in Pharmaceutical Salts: Properties, Selection, and Use, (Eds. P. H. Stahl and C. G. Wermuth), Wiley-VCH, 2008. The salts can be prepared in situ during the final isolation and purification of the compounds described herein or separately by reacting the free base group with a suitable organic acid.

The compounds of the invention may have ionizable groups so as to be capable of preparation as pharmaceutically acceptable salts. These salts may be acid addition salts involving inorganic or organic acids or the salts may, in the case of acidic forms of the compounds of the invention be prepared from inorganic or organic bases. Frequently, the compounds are prepared or used as pharmaceutically acceptable salts prepared as addition products of pharmaceutically acceptable acids or bases. Suitable pharmaceutically acceptable acids and bases are well-known in the art, such as hydrochloric, sulphuric, hydrobromic, acetic, lactic, citric, or tartaric acids for forming acid addition salts, and potassium hydroxide, sodium hydroxide, ammonium hydroxide, caffeine, various amines, and the like for forming basic salts. Methods for preparation of the appropriate salts are well-established in the art.

Representative acid addition salts include acetate, adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, fumarate, glucoheptonate, glycerophosphate, hemisulfate, heptonate, hexanoate, hydrobromide, hydrochloride, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, toluenesulfonate, undecanoate, valerate salts and the like. Representative alkali or alkaline earth metal salts include sodium, lithium, potassium, calcium, magnesium and the like, as well as nontoxic ammonium, quaternary ammonium, and amine cations, including, but not limited to ammonium, tetramethylammonium, tetraethylammonium, methylamine, dimethylamine, trimethylamine, triethylamine, and ethylamine.

“Predetermined level” as used herein, refers to a pre-specified particular level of one or more particular cell type, e.g., MDSCs such as monocytic and/or granulocytic MDSCs or activated T-cells. In some embodiments, a predetermined level is an absolute value or range. In some embodiments, a predetermined level is a relative value. In some embodiments, a predetermined level is the same as or different (e.g., higher or lower than) a level of one or more particular cell type in a reference, e.g., a reference tumor sample, or a level specified in a reference document such as a pharmaceutical specification.

In some embodiments, a predetermined level is an absolute level or range of one or more cell type in a sample. In some embodiments, a predetermined level is a level or range of one or more cell types in a sample relative to total level of cells in the sample. In some embodiments, a predetermined level is a level or range of one or more cell types in a sample relative to total level of cells in the sample. In some embodiments, a predetermined level is expressed as a percent.

“Progression-free survival” as used herein, refers to the length of time during and after medication or treatment during which the disease being treated (e.g., cancer) does not get worse.

“Proliferation” as used in this application involves reproduction or multiplication of similar forms (cells) due to constituting (cellular) elements.

By a “reference” is meant any useful reference used to compare protein or mRNA levels related to cancer. The reference can be any sample, standard, standard curve, or level that is used for comparison purposes. The reference can be a normal reference sample or a reference standard or level. A “reference sample” can be, for example, a control, e.g., a predetermined negative control value such as a “normal control” or a prior sample taken from the same subject; a sample from a normal healthy subject, such as a normal cell or normal tissue; a sample (e.g., a cell or tissue) from a subject not having cancer; a sample from a subject that has been treated for cancer (e.g., with an LXRβ agonist); or a sample of a purified protein (e.g., any described herein) at a known normal concentration. By “reference standard or level” is meant a value or number derived from a reference sample. A “normal control value” is a pre-determined value indicative of non-disease state, e.g., a value expected in a healthy control subject. Typically, a normal control value is expressed as a range (“between X and Y”), a high threshold (“no higher than X”), or a low threshold (“no lower than X”). A subject having a measured value within the normal control value for a particular biomarker is typically referred to as “within normal limits” for that biomarker. A normal reference standard or level can be a value or number derived from a normal subject not having cancer; or a subject that has been treated for cancer. In preferred embodiments, the reference sample, standard, or level is matched to the sample subject sample by at least one of the following criteria: age, weight, sex, disease stage, and overall health. A standard curve of levels of a purified protein, e.g., any described herein, within the normal reference range can also be used as a reference.

As used herein, “slowing the spread of metastasis” refers to reducing or stopping the formation of new loci; or reducing, stopping, or reversing the tumor load.

As used herein, “slowing the spread of migrating cancer” refers to reducing or stopping the formation of new loci; or reducing, stopping, or reversing the tumor load.

The term “subject,” as used herein, refers to a human or non-human animal (e.g., a mammal such as a non-human primate, horse, cow, or dog).

The term “substantially” refers to the qualitative condition of exhibiting total or near-total extent or degree of a characteristic or property of interest. One of ordinary skill in the biological arts will understand that biological and chemical phenomena rarely, if ever, go to completion and/or proceed to completeness or achieve or avoid an absolute result. The term “substantially” is therefore used herein to capture the potential lack of completeness inherent in many biological and chemical phenomena.

A “therapeutic regimen” refers to a dosing regimen whose administration across a relevant population is correlated with a desired or beneficial therapeutic outcome.

The term “treatment” (also “treat” or “treating”), in its broadest sense, refers to any administration of a substance (e.g., provided compositions) that partially or completely alleviates, ameliorates, relives, inhibits, delays onset of, reduces severity of, and/or reduces incidence of one or more symptoms, features, and/or causes of a particular disease, disorder, and/or condition. In some embodiments, such treatment may be administered to a subject who does not exhibit signs of the relevant disease, disorder and/or condition and/or of a subject who exhibits only early signs of the disease, disorder, and/or condition. Alternatively, or additionally, in some embodiments, treatment may be administered to a subject who exhibits one or more established signs of the relevant disease, disorder and/or condition. In some embodiments, treatment may be of a subject who has been diagnosed as suffering from the relevant disease, disorder, and/or condition. In some embodiments, treatment may be of a subject known to have one or more susceptibility factors that are statistically correlated with increased risk of development of the relevant disease, disorder, and/or condition.

As used herein, “tumor seeding” refers to the spillage of tumor cell clusters and their subsequent growth as malignant implants at a site other than the site of the original tumor.

The term “PD-1 inhibitor,” as used herein, refers to a compound such as an antibody capable of inhibiting the activity of the protein that in humans is encoded by the PDCD1 gene. Known PD-1 inhibitors include nivolumab, pembrolizumab, pidilizumab, BMS 936559, and MPDL3280A.

The term “PD-L1 inhibitor,” as used herein, refers to a compound such as an antibody capable of inhibiting the activity of the protein that in humans is encoded by the CD274 gene. Known PD-L1 inhibitors include atezolizumab and MEDI4736.

The term “CTLA-4 inhibitor,” as used herein, refers to a compound such as an antibody capable of inhibiting the activity of the protein that in humans is encoded by the CTLA4 gene. Known CTLA-4 inhibitors include ipilimumab.

The term “CSF-1R inhibitors,” as used herein refers to a compound such as an antibody capable of inhibiting the activity of the protein that in humans is encoded by the CSF1R gene. Known CSF-1R inhibitors include pexidartinib and AZD6495.

The term “IDO inhibitor,” as used herein, refers to a compound such as an antibody capable of inhibiting the activity of the protein that in humans is encoded by the IDO1 gene. Known IDO inhibitors include norharmane, rosmarinic acid, and alpha-methyl-tryptophan.

The term “A1 adenosine inhibitor,” as used herein, refers to a compound such as an antibody capable of inhibiting the activity of the protein that in humans is encoded by the ADORA1 gene. Known A1 adenosine inhibitors include 8-cyclopentyl-1,3-dimethylxanthine, 8-cyclopentyl-1,3-dipropylxanthine, 8-phenyl-1,3-dipropylxanthine, bamifylline, BG-9719, BG-9928, FK-453, FK-838, rolofylline, and N-0861.

The term “A2A adenosine inhibitor,” as used herein, refers to a compound such as an antibody capable of inhibiting the activity of the protein that in humans is encoded by the ADORA2A gene. Known A2A adenosine inhibitors include ATL-4444, istradefylline, MSX-3, preladenant, SCH-58261, SCH-412,348, SCH-442,416, ST-1535, VER-6623, VER-6947, VER-7835, viadenant, and ZM-241,385.

The term “A2B adenosine inhibitor,” as used herein, refers to a compound such as an antibody capable of inhibiting the activity of the protein that in humans is encoded by the ADORA2B gene. Known A2B adenosine inhibitors include ATL-801, CVT-6883, MRS-1706, MRS-1754, OSIP-339,391, PSB-603, PSB-0788, and PSB-1115.

The term “A3A adenosine inhibitor,” as used herein, refers to a compound such as an antibody capable of inhibiting the activity of the protein that in humans is encoded by the ADORA3 gene. Known A3A adenosine inhibitors include KF-26777, MRS-545, MRS-1191, MRS-1220, MRS-1334, MRS-1523, MRS-3777, MRE-3005-F20, MRE-3008-F20, PSB-11, OT-7999, VUF-5574, and SSR161421.

The term “arginase inhibitor,” as used herein, refers to a compound capable of inhibiting the activity of a protein that in humans is encoded by the ARG1 or ARG2 genes. Known arginase inhibitors include (2s)-(+)-amino-5-iodoacetamidopentanoic acid, NG-hydroxy-L-arginine, (2S)-(+)-amino-6-iodoacetamidohexanoic acid, and (R)-2-amino-6-borono-2-(2-(piperidin-1-yl)ethyl)hexanoic acid.

The term “HDAC inhibitor,” as used herein, refers to a compound such as an antibody that is capable of inhibiting the activity of the protein that is a member of the histone deacetylase class of enzymes, e.g., HDAC1, HDAC2, HDAC3, HDAC4, HDAC5, HDAC6, HDAC7, HDAC8, HDAC9, HDAC10, HDAC11, SIRT1, SIRT2, SIRT3, SIRT4, SIRT5, SIRT6, and SIRT7. Known HDAC inhibitors include valproic acid, SAHA, and romidepsin.

Unless otherwise defined, all technical and scientific terms used herein have the same meaning as commonly understood by one of ordinary skill in the art to which this invention belongs. Methods and materials are described herein for use in the present disclosure; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

The details of one or more embodiments of the invention are set forth in the description below. Other features, objects, and advantages of the invention will be apparent from the description and from the claims.

### DETAILED DESCRIPTION OF THE INVENTION

The invention features methods of treating cancer by administering 2-[3-[(3R)-3-[[2-chloro-3-(trifluoromethyl)phenyl]methyl-(2,2-diphenylethyl)amino]butoxy]phenyl]acetic acid at least once daily for four to six days followed by one to three days without administration of 2-[3-[(3R)-3-[[2-chloro-3-(trifluoromethyl)phenyl]methyl-(2,2-diphenylethyl)amino]butoxy]phenyl]acetic acid. The inventors have discovered that this dosing schedule surprisingly results in similar efficacy to daily dosing, but a decreased risk of adverse events, e.g., neutropenia.

### 2-[3-[(3R)-3-[[2-chloro-3-(trifluoromethyl)phenyl]methyl-(2,2-diphenylethyl)amino]butoxy]phenyl]acetic acid

2-[3-[(3R)-3-[[2-chloro-3-(trifluoromethyl)phenyl]methyl-(2,2-diphenylethyl)amino]butoxy]phenyl]acetic acid is an LXRβ agonist having the structure:

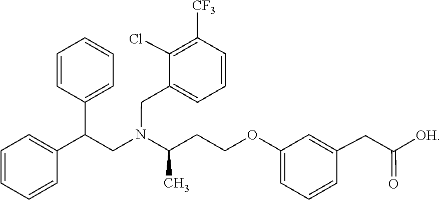 [SMILES: C[C@H](CCOc1cccc(CC(=O)O)c1)N(Cc1cccc(C(F)(F)F)c1Cl)CC(c1ccccc1)c1ccccc1]

2-[3-[(3R)-3-[[2-chloro-3-(trifluoromethyl)phenyl]methyl-(2,2-diphenylethyl)amino]butoxy]phenyl]acetic acid mediated activation of the LXR signaling pathway has been shown to induce expression of ApoE, which functions as a tumor suppressor gene by virtue of its ability to regulate key features of tumorigenesis. 2-[3-[(3R)-3-[[2-chloro-3-(trifluoromethyl)phenyl]methyl-(2,2-diphenylethyl)amino]butoxy]phenyl]acetic acid has a higher specificity for the LXRβ isoform. These features include suppression of cancer cell invasion (˜45% in vitro), inhibition of endothelial recruitment (˜50% in vitro) and the reduction of MDSCs in circulation (˜40%) and in tumors (>60%). In in vitro studies, 2-[3-[(3R)-3-[[2-chloro-3-(trifluoromethyl)phenyl]methyl-(2,2-diphenylethyl)amino]butoxy]phenyl]acetic acid induced ApoE gene expression by 3-fold in cancer cells and up to 40-fold in human peripheral blood mononuclear cells (hPBMCs) compared to control cells. The EC50 of 2-[3-[(3R)-3-[[2-chloro-3-(trifluoromethyl)phenyl]methyl-(2,2-diphenylethyl)amino]butoxy]phenyl]acetic acid for ApoE induction was 385 nM in cancer cells and 271 nM in hPBMCs.

In syngeneic and human xenograft mouse tumor models of melanoma (harboring different genetic backgrounds), glioblastoma, TNBC, ovarian cancer, and lung cancer, RGX-104 inhibited primary tumor growth by 48-95%. Extent of tumor growth inhibition varied with model. In a mouse model of TNBC metastasis, 2-[3-[(3R)-3-[[2-chloro-3-(trifluoromethyl)phenyl]methyl-(2,2-diphenylethyl)amino]butoxy]phenyl]acetic acid inhibited metastatic spread of cancer cells by ˜9-fold. Moreover, the anti-tumor activity of 2-[3-[(3R)-3-[[2-chloro-3-(trifluoromethyl)phenyl]methyl-(2,2-diphenylethyl)amino]butoxy]phenyl]acetic acid in combination with an anti-PD-1 antibody inhibited tumor growth by >80% in a syngeneic mouse melanoma model that is otherwise not responsive to anti-PD-1 antibody. Furthermore, inhibition of tumor growth in the same syngeneic mouse melanoma model was superior when the mice received combination therapy of 2-[3-[(3R)-3-[[2-chloro-3-(trifluoromethyl)phenyl]methyl-(2,2-diphenylethyl)amino]butoxy]phenyl]acetic acid and anti-CTLA-4 antibody relative to either therapy alone. Similarly, in a syngeneic mouse melanoma model, 2-[3-[(3R)-3-[[2-chloro-3-(trifluoromethyl)phenyl]methyl-(2,2-diphenylethyl)amino]butoxy]phenyl]acetic acid demonstrated superior anti-tumor efficacy in combination with dacarbazine (>80%), compared to either treatment alone. In tumor growth studies, minimum efficacious doses ranged from 25-40 mg/kg/day administered orally (PO) resulting in exposures that ranged from 10,000-50,000 ng-h/mL.

In safety pharmacology assessments, 2-[3-[(3R)-3-[[2-chloro-3-(trifluoromethyl)phenyl]methyl-(2,2-diphenylethyl)amino]butoxy]phenyl]acetic acid produced a significant increase, but not inhibition of human ether-n-go-go-related gene (hERG) channel conductance in an in vitro hERG assay. There were no 2-[3-[(3R)-3-[[2-chloro-3-(trifluoromethyl)phenyl]methyl-(2,2-diphenylethyl)amino]butoxy]phenyl]acetic acid-related effects on qualitative electrocardiogram (ECG) parameters (PR or QTc intervals or QRS duration) in dogs, but there was a dose related decrease in mean heart rate at the Day 1 post-dose interval that was significantly different in females following the 150 (stepped down to 100) mg/kg/day dose. This change was not observed during the recovery period and was not considered adverse. Furthermore, no adverse effects were noted during the neurobehavioral functional observation battery (FOB) or respiratory evaluations in rats. Given the favorable safety profile of 2-[3-[(3R)-3-[[2-chloro-3-(trifluoromethyl)phenyl]methyl-(2,2-diphenylethyl)amino]butoxy]phenyl]acetic acid at the highest doses tested in the repeat dose toxicity studies, the potential for cardiovascular, respiratory or central nervous system (CNS) system effects is considered low.

In oral PK studies, 2-[3-[(3R)-3-[[2-chloro-3-(trifluoromethyl)phenyl]methyl-(2,2-diphenylethyl) amino]butoxy]phenyl]acetic acid was well absorbed in CD-1 mice with a calculated absolute oral bioavailability (% F) often >100%, indicative of possible enterohepatic recycling of parent compound. The time to maximum plasma concentration (Tmax) was similar for males and females and ranged from 2 to 8 h. The mean apparent oral half-life (t½) ranged from 6.5-8 h in mice. There was a significant food effect showing that 2-[3-[(3R)-3-[[2-chloro-3-(trifluoromethyl)phenyl]methyl-(2,2-diphenylethyl)amino]butoxy]phenyl]acetic acid plasma concentrations were >2-fold higher when administered to mice in the fed state. In Sprague-Dawley rats, the combined, mean % F (following a 30 mg/kg oral dose of 2-[3-[(3R)-3-[[2-chloro-3-(trifluoromethyl)phenyl]methyl-(2,2-diphenylethyl)amino]butoxy]phenyl]acetic acid) was moderate (˜31%), and Tmax was similar for males and females, ranging from 4 to 8 h. 2-[3-[(3R)-3-[[2-chloro-3-(trifluoromethyl)phenyl]methyl-(2,2-diphenylethyl)amino]butoxy]phenyl]acetic acid was cleared at a lower rate in female rats compared to males, which resulted in higher systemic exposure to 2-[3-[(3R)-3-[[2-chloro-3-(trifluoromethyl)phenyl]methyl-(2,2-diphenylethyl)amino]butoxy]phenyl]acetic acid in females at all dose levels tested. The mean apparent oral t½ in female rats was 6.5 h (not calculable in males). In male Beagle dogs given an oral dose of 2-[3-[(3R)-3-[[2-chloro-3-(trifluoromethyl)phenyl]methyl-(2,2-diphenylethyl)amino]butoxy]phenyl]acetic acid, Tmax ranged from 4-8 h. The mean % F was moderate (18-30% depending on dose and formulation) and mean apparent oral t½ ranged from 5-6.7 h. In Cynomolgus monkeys, mean % F was low to moderate (6-19% depending on dose and formulation). Following an oral dose, monkeys had a mean Tmax of 4 h. Mean oral t½ ranged from 5.5-8 h.

2-[3-[(3R)-3-[[2-chloro-3-(trifluoromethyl)phenyl]methyl-(2,2-diphenylethyl)amino]butoxy]phenyl]acetic acid is subjected to phase I and phase II metabolism, which includes oxidation, dealkylation, glucuronidation and combinations thereof. In vitro, 2-[3-[(3R)-3-[[2-chloro-3-(trifluoromethyl)phenyl]methyl-(2,2-diphenylethyl)amino]butoxy]phenyl]acetic acid is metabolized predominantly by the cytochrome P450 (CYP) isoform CYP3A4, but it is also a substrate for CYP2E1, CYP2C9, CYP2C19, and possibly CYP2J2. While 2-[3-[(3R)-3-[[2-chloro-3-(trifluoromethyl)phenyl]methyl-(2,2-diphenylethyl)amino]butoxy]phenyl]acetic acid is not a strong inhibitor of any human CYP450s in vitro, it is a moderate inhibitor of CYP2C8 (7.5 μM 50% inhibitory concentration [IC50]) and a weak inhibitor of 2B6 (15 μM IC50). RGX-104 very weakly inhibited 1A1, 2A6, 2C9, 2C19, 2D6, 2E1, and 3A4 in vitro, but CYP3A time-dependent inhibition (TDI) was demonstrated in vitro using testosterone as the substrate. Induction of CYP3A by 2-[3-[(3R)-3-[[2-chloro-3-(trifluoromethyl)phenyl]methyl-(2,2-diphenylethyl)amino]butoxy]phenyl]acetic acid was demonstrated in primary cultures of cryopreserved hepatocytes (2 donors) and the potential of 2-[3-[(3R)-3-[[2-chloro-3-(trifluoromethyl)phenyl]methyl-(2,2-diphenylethyl)amino]butoxy]phenyl]acetic acid to induce CYP2B6 cannot be ruled out (1 concentration in ⅓ donors was induced >2-fold). 2-[3-[(3R)-3-[[2-chloro-3-(trifluoromethyl)phenyl]methyl-(2,2-diphenylethyl)amino]butoxy]phenyl]acetic acid did not induce CYP1A2. In efflux transporters, 2-[3-[(3R)-3-[[2-chloro-3-(trifluoromethyl)phenyl]methyl-(2,2-diphenylethyl)amino]butoxy]phenyl]acetic acid does not inhibit P-glucoprotein (P-gp) but does inhibit breast cancer resistance protein (BCRP) transport in vitro (55% at 5 μM). 2-[3-[(3R)-3-[[2-chloro-3-(trifluoromethyl)phenyl]methyl-(2,2-diphenylethyl)amino]butoxy]phenyl]acetic acid is a potent inhibitor of the uptake transporter organic anion transporting polypeptide (OATP) 1B1 in vitro (0.099 μM IC50). 2-[3-[(3R)-3-[[2-chloro-3-(trifluoromethyl)phenyl]methyl-(2,2-diphenylethyl)amino]butoxy]phenyl]acetic acid also appears to be a moderate inhibitor of OATP1B3 (3.7 μM IC50). 2-[3-[(3R)-3-[[2-chloro-3-(trifluoromethyl)phenyl]methyl-(2,2-diphenylethyl)amino]butoxy]phenyl]acetic acid only weakly inhibited OAT1, OAT3, and OCT2 in vitro with inhibition less than 50% at 50 μM.

Potential risks with RGX-104 in the clinical setting based on animal toxicology studies of 2-[3-[(3R)-3-[[2-chloro-3-(trifluoromethyl)phenyl]methyl-(2,2-diphenylethyl)amino]butoxy]phenyl]acetic acid include elevations in serum cholesterol and TG, neutropenia/leukopenia, nausea and/or vomiting, elevations in liver enzymes, development or worsening of cataracts, cardiac rhythm disturbances and/or reduced cardiac function, harderian gland adenocarcinoma, and/or generalized edema.

### Nivolumab

Nivolumab is a fully human immunoglobulin (Ig) G4 monoclonal antibody directed against the negative immunoregulatory human cell surface receptor programmed death-1 (PD-1) with immune checkpoint inhibitory and antineoplastic activities. Nivolumab binds to and blocks the activation of PD-1, an Ig superfamily transmembrane protein, by its ligands programmed cell death ligand 1 (PD-L1), overexpressed on certain cancer cells, and programmed cell death ligand 2 (PD-L2), which is primarily expressed on Antigen Presenting Cells. This results in the activation of T-cells and cell-mediated immune responses against tumor cells or pathogens. Activated PD-1 negatively regulates T-cell activation and plays a key role in tumor evasion from host immunity. The nivolumab dose will be 240 mg administered as an intravenous infusion over 60 minutes on Days 1 and 15 of each 28-day cycle.

There is potential for overlapping toxicities between 2-[3-[(3R)-3-[[2-chloro-3-(trifluoromethyl)phenyl]methyl-(2,2-diphenylethyl)amino]butoxy]phenyl]acetic acid and nivolumab. Specifically, a DLT of Grade 4 neutropenia has been seen on 2-[3-[(3R)-3-[[2-chloro-3-(trifluoromethyl)phenyl]methyl-(2,2-diphenylethyl)amino]butoxy]phenyl]acetic acid single agent therapy and myelosuppression may be observed with nivolumab treatment. The pharmacological effects of 2-[3-[(3R)-3-[[2-chloro-3-(trifluoromethyl)phenyl]methyl-(2,2-diphenylethyl)amino]butoxy]phenyl]acetic acid include modulation of sterol biosynthesis. Consequently, hyperlipidemia has been observed in subjects treated with 2-[3-[(3R)-3-[[2-chloro-3-(trifluoromethyl)phenyl]methyl-(2,2-diphenylethyl)amino]butoxy]phenyl]acetic acid, which has also been reported in subjects treated with nivolumab. Liver function abnormalities have been observed in pre-clinical toxicity studies with 2-[3-[(3R)-3-[[2-chloro-3-(trifluoromethyl)phenyl]methyl-(2,2-diphenylethyl)amino]butoxy]phenyl]acetic acid and immune-mediated hepatitis has been observed with nivolumab treatment.

### Ipilimumab

Ipilimumab is a recombinant human IgG1 kappa monoclonal antibody that binds to the cytotoxic T-lymphocyte-associated protein 4 (CTLA-4). CTLA-4 is a negative regulator of T-cell activity. By binding to CTLA-4, ipilimumab blocks the interaction of CTLA-4 with its ligands, CD80/CD86. Blockade of CTLA-4 has been shown to augment T-cell activation and proliferation, including the activation and proliferation of tumor infiltrating T-effector cells. Inhibition of CTLA-4 signaling can also reduce T-regulatory cell function, which may contribute to a general increase in T cell responsiveness, including the anti-tumor immune response.

In some embodiments, the ipilimumab dose is 3 mg/kg administered as an IV infusion on Day 1 of each 28-day cycle for a maximum of 4 doses.

There is potential for overlapping toxicities between 2-[3-[(3R)-3-[[2-chloro-3-(trifluoromethyl)phenyl]methyl-(2,2-diphenylethyl)amino]butoxy]phenyl]acetic acid and ipilimumab. Liver function abnormalities have been observed in pre-clinical toxicity studies with 2-[3-[(3R)-3-[[2-chloro-3-(trifluoromethyl)phenyl]methyl-(2,2-diphenylethyl)amino]butoxy]phenyl]acetic acid; immune-mediated hepatitis has been observed with ipilimumab treatment.

### Docetaxel

Docetaxel is an antineoplastic agent belonging to the taxoid family. It is prepared by a semisynthesis beginning with a precursor extracted from the renewable needle biomass of yew plants. The chemical name for docetaxel is (2R,3S)—N-carboxy-3-phenylisoserine,N-tert-butyl ester, 13-ester with 5b-20-epoxy-1,2a,4,7b, 10b, 13a-hexahydroxytax-11-en-9-one 4-acetate 2-benzoate, trihydrate.

In some embodiments, the docetaxel is administered as an IV infusion on days 1, 8, and 15 of each 28-day cycle. In some embodiments, the docetaxel dose was 35 mg/m2. In some embodiments, 28 mg/m2 is the docetaxel dose.

There is potential for overlapping toxicities between 2-[3-[(3R)-3-[[2-chloro-3-(trifluoromethyl)phenyl]methyl-(2,2-diphenylethyl)amino]butoxy]phenyl]acetic acid and docetaxel. Specifically, a DLT of Grade 4 neutropenia has been seen on 2-[3-[(3R)-3-[[2-chloro-3-(trifluoromethyl)phenyl]methyl-(2,2-diphenylethyl)amino]butoxy]phenyl]acetic acid single agent therapy and myelosuppression may be observed with docetaxel treatment. Liver function abnormalities have been observed in pre-clinical toxicity studies with 2-[3-[(3R)-3-[[2-chloro-3-(trifluoromethyl)phenyl]methyl-(2,2-diphenylethyl)amino]butoxy]phenyl]acetic acid; hepatotoxicity has been observed with docetaxel treatment.

### Pembrolizumab

Pembrolizumab is a programmed death receptor-1 (PD 1)-blocking antibody. Pembrolizumab is a humanized monoclonal IgG4 kappa antibody with an approximate molecular weight of 149 kDa. Pembrolizumab is produced in recombinant Chinese hamster ovary (CHO) cells.

In some embodiments, pembrolizumab is administered as a dose of 200 mg using a 30 minutes IV infusion on Day 1 of each 21 days cycle after all procedures and assessments have been completed and prior to the administration of other drugs, and with a gap of 30 minutes between the administration of next drug.

### Carboplatin

The chemical name for carboplatin, USP is platinum, diammine[1,1-cyclobutanedicarboxylato(2-)-O,O′]-, (SP-4-2). Carboplatin, USP is a crystalline powder. It is soluble in water at a rate of approximately 14 mg/mL, and the pH of a 1% solution is 5-7. It is virtually insoluble in ethanol, acetone, and dimethylacetamide. Carboplatin produces predominantly interstrand DNA cross-links rather than DNA-protein cross-links. This effect is apparently cell cycle nonspecific. Carboplatin induce equal numbers of drug-DNA cross-links, causing equivalent lesions and biological effects.

In some embodiments, the initial dose of carboplatin injection is determined by the use of mathematical formulae, which is based on a subject's pre-existing renal function or renal function and desired platelet nadir (as renal excretion is the major route of elimination for carboplatin). The use of dosing formulae, as compared to empirical dose calculation based on body surface area, allows compensation for subject variations in pretreatment renal function that might otherwise result in either underdosing (in subjects with above average renal function) or overdosing (in subjects with impaired renal function).

A simple formula for calculating dosage, based upon a subject's glomerular filtration rate (GFR in mL/min) and carboplatin injection target area under the concentration versus time curve (AUC in mg/mL·min), has been proposed by Calvert. In these studies, GFR was measured by Cr-EDTA clearance. The Calvert formula for carboplatin dosing is as follows:

Total Dose (mg)=(target AUC)×(GFR+25)

Note that with this formula, the total dose of carboplatin is calculated in mg, not mg/m2. The target AUC of 4 mg/mL·min to 6 mg/mL·min using single agent carboplatin appears to provide the most appropriate dose range in previously treated subjects. This study also showed a trend between the AUC of single agent carboplatin administered to previously treated subjects and the likelihood of developing toxicity.

### Pemetrexed

Pemetrexed (for injection) is a folate analog metabolic inhibitor. The drug substance, pemetrexed disodium heptahydrate, has the chemical name L-glutamic acid, N-[4-[2-(2-amino-4,7-dihydro-4-oxo-1H-pyrrolo[2,3-d]pyrimidin-5-yl)ethyl]benzoyl]-, disodium salt, heptahydrate with a molecular formula of C20H119N5Na2O6.7H2O and a molecular weight of 597.49.

Pemetrexed exerts its antineoplastic activity by disrupting the folate-dependent metabolic processes essential for cell replication. In vitro studies have shown that pemetrexed behaves as a multitargeted antifolate by inhibiting thymidylate synthase (TS), dihydrofolate reductase (DHFR), and glycinamide ribonucleotide formyltransferase (GARFT) which are crucial for the de novo biosynthesis of thymidine and purine nucleotides. Polyglutamated metabolites of pemetrexed with prolonged intracellular half-life result in prolonged pemetrexed drug action in malignant cells.

In some embodiments, the pemetrexed dose is 500 mg/m2 on Day 1 of each 21-day cycle for a maximum of 4 cycles. In some embodiments, subjects treated with pemetrexed must be instructed to take folic acid and vitamin B12 as a prophylactic measure to reduce treatment-related hematologic and GI toxicity. In some embodiments, subjects may also be prescribed with corticosteroids to take 2 times a day for 3 days, beginning the day before each treatment with pemetrexed.

There is potential for overlapping toxicities between 2-[3-[(3R)-3-[[2-chloro-3-(trifluoromethyl)phenyl]methyl-(2,2-diphenylethyl)amino]butoxy]phenyl]acetic acid, pemetrexed, and carboplatin. Specifically, a DLT of Grade 4 neutropenia has been seen on 2-[3-[(3R)-3-[[2-chloro-3-(trifluoromethyl)phenyl]methyl-(2,2-diphenylethyl)amino]butoxy]phenyl]acetic acid single agent therapy and myelosuppression may be observed with pemetrexed combination treatment with 2-[3-[(3R)-3-[[2-chloro-3-(trifluoromethyl)phenyl]methyl-(2,2-diphenylethyl)amino]butoxy]phenyl]acetic acid and carboplatin.

### Adverse Events

An adverse event (AE) is any untoward medical occurrence in a subject or clinical investigation subject administered a pharmaceutical product, and which does not necessarily have to have a causal relationship with this treatment. An AE can therefore be any unfavorable and unintended sign (including abnormal laboratory findings), symptom, or disease temporally associated with the use of an investigational product, whether or not related to the investigational product.

Death and progressive disease (PD) are not considered AEs. Death is considered an outcome of one or more primary AEs, and PD is considered a worsening of underlying disease. Preexisting conditions (present before the start of the AE collection period) are considered concurrent medical conditions and not AEs. However, a worsening or complication of such a concurrent condition, the worsening or complication is an AE.

An AE or suspected adverse reaction is considered serious if it results in death; is life threatening, i.e., the subject was at immediate risk of death from the reaction as it occurred but does not include a reaction which hypothetically might have caused death had it occurred in a more severe form; requires in-subject hospitalization or prolongation of existing hospitalization; results in persistent or significant disability/incapacity; is a congenital anomaly/birth defect; or is an important medical event.

### Methods of Treatment

The methods described here can be used to treat cancer.

Treating cancer can result in a reduction in size or volume of a tumor. For example, after treatment, tumor size is reduced by 5% or greater (e.g., 10%, 20%, 30%, 40%, 50%, 60%, 70%, 80%, 90% or greater) relative to its size prior to treatment. Size of a tumor may be measured by any reproducible means of measurement. The size of a tumor may be measured as a diameter of the tumor or by any reproducible means of measurement.

Treating cancer may further result in a decrease in number of tumors. For example, after treatment, tumor number is reduced by 5% or greater (e.g., 10%, 20%, 30%, 40%, 50%, 60%, 70%, 80%, 90% or greater) relative to number prior to treatment. Number of tumors may be measured by any reproducible means of measurement. The number of tumors may be measured by counting tumors visible to the naked eye or at a specified magnification (e.g., 2×, 3×, 4×, 5×, 10×, or 50×).

Treating cancer can result in a decrease in number of metastatic nodules in other tissues or organs distant from the primary tumor site. For example, after treatment, the number of metastatic nodules is reduced by 5% or greater (e.g., 10%, 20%, 30%, 40%, 50%, 60%, 70%, 80%, 90% or greater) relative to number prior to treatment. The number of metastatic nodules may be measured by any reproducible means of measurement. The number of metastatic nodules may be measured by counting metastatic nodules visible to the naked eye or at a specified magnification (e.g., 2×, 10×, or 50×).

Treating cancer can result in an increase in average survival time of a population of subjects treated according to the present invention in comparison to a population of untreated subjects. For example, the average survival time is increased by more than 30 days (more than 60 days, 90 days, or 120 days). An increase in average survival time of a population may be measured by any reproducible means. An increase in average survival time of a population may be measured, for example, by calculating for a population the average length of survival following initiation of treatment with the compound of the invention. An increase in average survival time of a population may also be measured, for example, by calculating for a population the average length of survival following completion of a first round of treatment with the compound of the invention.

Treating cancer can also result in a decrease in the mortality rate of a population of treated subjects in comparison to an untreated population. For example, the mortality rate is decreased by more than 2% (e.g., more than 5%, 10%, or 25%). A decrease in the mortality rate of a population of treated subjects may be measured by any reproducible means, for example, by calculating for a population the average number of disease-related deaths per unit time following initiation of treatment with the compound of the invention. A decrease in the mortality rate of a population may also be measured, for example, by calculating for a population the average number of disease-related deaths per unit time following completion of a first round of treatment with the compound of the invention.

Treating cancer can also result in an increased average progression-free survival time of a population of treated subjects in comparison to an untreated population. For example, the average progression-free survival time is increased by more than 30 days (more than 60 days, 90 days, or 120 days). An increase in average progression-free survival time of a population may be measured by any reproducible means. An increase in average progression-free survival time of a population may be measured, for example, by calculating for a population the average length of progression-free survival following initiation of treatment with the compound of the invention. An increase in average progression-free survival time of a population may also be measured, for example, by calculating for a population the average length of progression-free survival following completion of a first round of treatment with the compound of the invention.

In some embodiments, the methods described herein may be useful for the treatment of infections such as bacterial infections, parasitic infections, or fungal infections. Compounds of the present invention may be administered by any appropriate route for treatment or prophylactic treatment of a disease or condition associated with an infection. These may be administered to humans, domestic pets, livestock, or other animals with a pharmaceutically acceptable diluent, carrier, or excipient. Administration may be topical, parenteral, intravenous, intra-arterial, subcutaneous, intramuscular, intracranial, intraorbital, ophthalmic, intraventricular, intracapsular, intraspinal, intracisternal, intraperitoneal, intranasal, aerosol, by suppositories, or oral administration.

### Compositions

Within the scope of this invention is a composition that contains a suitable carrier and one or more of the therapeutic agents described above. The composition can be a pharmaceutical composition that contains a pharmaceutically acceptable carrier, a dietary composition that contains a dietarily acceptable suitable carrier, or a cosmetic composition that contains a cosmetically acceptable carrier.

The term “pharmaceutical composition” refers to the combination of an active agent with a carrier, inert or active, making the composition especially suitable for diagnostic or therapeutic use in vivo or ex vivo. A “pharmaceutically acceptable carrier,” after administered to or upon a subject, does not cause undesirable physiological effects. The carrier in the pharmaceutical composition must be “acceptable” also in the sense that it is compatible with the active ingredient and can be capable of stabilizing it. One or more solubilizing agents can be utilized as pharmaceutical carriers for delivery of an active compound. Examples of a pharmaceutically acceptable carrier include, but are not limited to, biocompatible vehicles, adjuvants, additives, and diluents to achieve a composition usable as a dosage form. Examples of other carriers include colloidal silicon oxide, magnesium stearate, cellulose, sodium lauryl sulfate, and D&C Yellow #10.

As used herein, the term “pharmaceutically acceptable salt” refers to those salts which are, within the scope of sound medical judgment, suitable for use in contact with the tissues of humans and lower animals without undue toxicity, irritation, or allergic response, and are commensurate with a reasonable benefit/risk ratio. Pharmaceutically acceptable salts of amines, carboxylic acids, and other types of compounds, are well known in the art. For example, S. M. Berge, et al. describe pharmaceutically acceptable salts in detail in J. Pharmaceutical Sciences, 66: 1-19 (1977), incorporated herein by reference. The salts can be prepared in situ during the final isolation and purification of the compounds of the invention, or separately by reacting a free base or free acid function with a suitable reagent, as described generally below. For example, a free base function can be reacted with a suitable acid. Furthermore, where the compounds of the invention carry an acidic moiety, suitable pharmaceutically acceptable salts thereof may, include metal salts such as alkali metal salts, e.g. sodium or potassium salts; and alkaline earth metal salts, e.g. calcium or magnesium salts. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts, include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, and valerate salts. Representative alkali or alkaline earth metal salts include sodium, lithium, potassium, calcium, and magnesium. Further pharmaceutically acceptable salts include, when appropriate, nontoxic ammonium, quaternary ammonium, and amine cations formed using counterions such as halide, hydroxide, carboxylate, sulfate, phosphate, nitrate, lower alkyl sulfonate and aryl sulfonate.

As described above, the pharmaceutical compositions of the present invention additionally include a pharmaceutically acceptable carrier, which, as used herein, includes any and all solvents, diluents, or other liquid vehicle, dispersion or suspension aids, surface active agents, isotonic agents, thickening or emulsifying agents, preservatives, solid binders, and lubricants, as suited to the particular dosage form desired. Remington's Pharmaceutical Sciences, Sixteenth Edition, E. W. Martin (Mack Publishing Co., Easton, Pa., 1980) discloses various carriers used in formulating pharmaceutical compositions and known techniques for the preparation thereof. Except insofar as any conventional carrier medium is incompatible with the compounds of the invention, such as by producing any undesirable biological effect or otherwise interacting in a deleterious manner with any other component(s) of the pharmaceutical composition, its use is contemplated to be within the scope of this invention. Some examples of materials which can serve as pharmaceutically acceptable carriers include, but are not limited to, sugars such as lactose, glucose and sucrose; starches such as corn starch and potato starch; cellulose and its derivatives such as sodium carboxymethyl cellulose, ethyl cellulose and cellulose acetate; powdered tragacanth; malt; gelatine; talc; excipients such as cocoa butter and suppository waxes; oils such as peanut oil, cottonseed oil; safflower oil, sesame oil; olive oil; corn oil and soybean oil; glycols; such as propylene glycol; esters such as ethyl oleate and ethyl laurate; agar; natural and synthetic phospholipids, such as soybean and egg yolk phosphatides, lecithin, hydrogenated soy lecithin, dimyristoyl lecithin, dipalmitoyl lecithin, distearoyl lecithin, dioleoyl lecithin, hydroxylated lecithin, lysophosphatidylcholine, cardiolipin, sphingomyelin, phosphatidylcholine, phosphatidyl ethanolamine, diastearoyl phosphatidylethanolamine (DSPE) and its pegylated esters, such as DSPE-PEG750 and, DSPE-PEG2000, phosphatidic acid, phosphatidyl glycerol and phosphatidyl serine. Commercial grades of lecithin which are preferred include those which are available under the trade name Phosal® or Phospholipon® and include Phosal 53 MCT, Phosal 50 PG, Phosal 75 SA, Phospholipon 90H, Phospholipon 90G and Phospholipon 90 NG; soy-phosphatidylcholine (SoyPC) and DSPE-PEG2000 are particularly preferred; buffering agents such as magnesium hydroxide and aluminum hydroxide; alginic acid; pyrogen-free water; isotonic saline; Ringer's solution; ethyl alcohol, and phosphate buffer solutions, as well as other non-toxic compatible lubricants such as sodium lauryl sulfate and magnesium stearate, as well as coloring agents, releasing agents, coating agents, sweetening, flavoring and perfuming agents, preservatives and antioxidants can also be present in the composition, according to the judgment of the formulator.

The above-described composition, in any of the forms described above, can be used for treating melanoma, or any other disease or condition described herein. An effective amount refers to the amount of an active compound/agent that is required to confer a therapeutic effect on a treated subject. Effective doses will vary, as recognized by those skilled in the art, depending on the types of diseases treated, route of administration, excipient usage, and the possibility of co-usage with other therapeutic treatment.

A pharmaceutical composition of this invention can be administered parenterally, orally, nasally, rectally, topically, or buccally. The term “parenteral” as used herein refers to subcutaneous, intracutaneous, intravenous, intramuscular, intraarticular, intraarterial, intrasynovial, intrasternal, intrathecal, intralesional, or intracranial injection, as well as any suitable infusion technique.

A sterile injectable composition can be a solution or suspension in a non-toxic parenterally acceptable diluent or solvent. Such solutions include, but are not limited to, 1,3-butanediol, mannitol, water, Ringer's solution, and isotonic sodium chloride solution. In addition, fixed oils are conventionally employed as a solvent or suspending medium (e.g., synthetic mono- or diglycerides). Fatty acid, such as, but not limited to, oleic acid and its glyceride derivatives, are useful in the preparation of injectables, as are natural pharmaceutically acceptable oils, such as, but not limited to, olive oil or castor oil, polyoxyethylated versions thereof. These oil solutions or suspensions also can contain a long chain alcohol diluent or dispersant such as, but not limited to, carboxymethyl cellulose, or similar dispersing agents. Other commonly used surfactants, such as, but not limited to, Tweens or Spans or other similar emulsifying agents or bioavailability enhancers, which are commonly used in the manufacture of pharmaceutically acceptable solid, liquid, or other dosage forms also can be used for the purpose of formulation.

A composition for oral administration can be any orally acceptable dosage form including capsules, tablets, emulsions and aqueous suspensions, dispersions, and solutions. In the case of tablets, commonly used carriers include, but are not limited to, lactose and corn starch. Lubricating agents, such as, but not limited to, magnesium stearate, also are typically added. For oral administration in a capsule form, useful diluents include, but are not limited to, lactose and dried corn starch. When aqueous suspensions or emulsions are administered orally, the active ingredient can be suspended or dissolved in an oily phase combined with emulsifying or suspending agents. If desired, certain sweetening, flavoring, or coloring agents can be added.

Pharmaceutical compositions for topical administration according to the described invention can be formulated as solutions, ointments, creams, suspensions, lotions, powders, pastes, gels, sprays, aerosols, or oils. Alternatively, topical formulations can be in the form of patches or dressings impregnated with active ingredient(s), which can optionally include one or more excipients or diluents. In some preferred embodiments, the topical formulations include a material that would enhance absorption or penetration of the active agent(s) through the skin or other affected areas.

A topical composition contains a safe and effective amount of a dermatologically acceptable carrier suitable for application to the skin. A “cosmetically acceptable” or “dermatologically-acceptable” composition or component refers a composition or component that is suitable for use in contact with human skin without undue toxicity, incompatibility, instability, or allergic response. The carrier enables an active agent and optional component to be delivered to the skin at an appropriate concentration(s). The carrier thus can act as a diluent, dispersant, solvent, or the like to ensure that the active materials are applied to and distributed evenly over the selected target at an appropriate concentration. The carrier can be solid, semi-solid, or liquid. The carrier can be in the form of a lotion, a cream, or a gel, in particular one that has a sufficient thickness or yield point to prevent the active materials from sedimenting. The carrier can be inert or possess dermatological benefits. It also should be physically and chemically compatible with the active components described herein, and should not unduly impair stability, efficacy, or other use benefits associated with the composition.

### Combination Therapies

In some embodiments of the methods described herein, the pharmaceutical composition may further include an additional compound having antiproliferative activity.

It will also be appreciated that the compounds and pharmaceutical compositions of the present invention can be formulated and employed in combination therapies, that is, the compounds and pharmaceutical compositions can be formulated with or administered concurrently with, prior to, or subsequent to, one or more other desired therapeutics or medical procedures. The particular combination of therapies (therapeutics or procedures) to employ in a combination regimen will take into account compatibility of the desired therapeutics and/or procedures and the desired therapeutic effect to be achieved. It will also be appreciated that the therapies employed may achieve a desired effect for the same disorder, or they may achieve different effects (e.g., control of any adverse effects).

### EXAMPLES

### Example 1. Adverse Event Profile of Full and Reduced Regimens of 2-[3-[(3R)-3-[[2-chloro-3-(trifluoromethyl)phenyl]methyl-(2,2-diphenylethyl)amino]butoxy]phenyl]acetic acid

Protocol: Three subjects was administered Regimen 1 (Full): 35 mg/m² of docetaxel on days 1, 8, and 15 of every 28-day cycle and 80 mg of 2-[3-[(3R)-3-[[2-chloro-3-(trifluoromethyl)phenyl]methyl-(2,2-diphenylethyl)amino]butoxy]phenyl]acetic acid twice per day daily throughout treatment. Seven subjects was administered Regimen 2 (Reduced): 28 mg/m² of docetaxel on days 1, 8, and 15 of every 28-day cycle and 80 mg of 2-[3-[(3R)-3-[[2-chloro-3-(trifluoromethyl)phenyl]methyl-(2,2-diphenylethyl)amino]butoxy]phenyl]acetic acid twice per day for five continuous days followed by a 2 day rest period throughout treatment. One subject that was administered Regimen 2 received 35 mg/m² of docetaxel on days 1 and 80 mg of 2-[3-[(3R)-3-[[2-chloro-3-(trifluoromethyl)phenyl]methyl-(2,2-diphenylethyl)amino]butoxy]phenyl]acetic acid twice per day for the first seven days of treatment prior to switching to the reduced treatment schedule for the remainder of the 28-day cycle. This subject had a grade 1 white blood cell decrease during this cycle.

Results: The incidence of neutropenia and decreased white blood cell count for each group of subjects is shown in Table 1 below.

TABLE 1
Frequency of Selected Hematologic
Adverse Events by Total Cycles

| | Regimen 1 | Regimen 2 |
|---|---|---|
| Regimen/Events | (AE/Cycle %) | (AE/Cycle %) |
| # of subjects | 3 | 7 |
| # of total cycles | 3 | 20 |
| Neutropenia (All grades) | 2 (67%) | 1 (5%) |
| Decreased white blood | 2 (67%) | 4 (20%) |
| cell count (All grades) | | |

Cycle definition: Once subject enters the cycle it is considered a cycle; % calculation = # AEs/# of cycles forthat regimen

As shown in Table 1, the incidence of neutropenia and decreased white blood cell count was decreased for subjects that were treated with the reduced regimen compared to the subjects treated with the full regimen.

### Example 2. Efficacy Profile of Full and Reduced Regimens of 2-[3-[(3R)-3-[[2-chloro-3-(trifluoromethyl)phenyl]methyl-(2,2-diphenylethyl)amino]butoxy]phenyl]acetic acid

Protocol: Five groups of Xenograft A549 mice were treated and tumor volume tracked for twenty-two days. Group 01 was administered 0.9% Saline, 0 mg/kg cisplatin, Corn Oil, and 0 mg/kg 2-[3-[(3R)-3-[[2-chloro-3-(trifluoromethyl)phenyl]methyl-(2,2-diphenylethyl)amino]butoxy]phenyl]acetic acid; Group 02 was administered 2 mg/kg Cisplatin, Corn Oil, and 0 mg/kg 2-[3-[(3R)-3-[[2-chloro-3-(trifluoromethyl)phenyl]methyl-(2,2-diphenylethyl)amino]butoxy]phenyl]acetic acid; Group 03 was administered 0.9% Saline, 0 mg/kg cisplatin, and 50 mg/kg 2-[3-[(3R)-3-[[2-chloro-3-(trifluoromethyl)phenyl]methyl-(2,2-diphenylethyl)amino]butoxy]phenyl]acetic acid; Group 04 was administered 2 mg/kg cisplatin and 50 mg/kg 2-[3-[(3R)-3-[[2-chloro-3-(trifluoromethyl)phenyl]methyl-(2,2-diphenylethyl)amino]butoxy]phenyl]acetic acid twice per day daily for the entire treatment period; and Group 05 was administered 2 mg/kg cisplatin and 50 mg/kg 2-[3-[(3R)-3-[[2-chloro-3-(trifluoromethyl)phenyl]methyl-(2,2-diphenylethyl)amino]butoxy]phenyl]acetic acid twice per day for five continuous days followed by two days without for the entire treatment period.

Results: The mean tumor volume for each group of mice on day 0, day 3, day 6, day 9, day 12, day 15, and day 22 is shown in Table 2.

| Day | 0 | 3 | 6 | 9 | 12 | 15 | 19 | 22 |
|---|---|---|---|---|---|---|---|---|
| Group | 86.75 +/− | 179.47 +/− | 299.51 +/− | 411.57 +/− | 587.29 +/− | 809.85 +/− | 1441.22 +/− | 1805.36 +/− |
| 01 | 1.53 | 5.60 | 8.78 | 42.18 | 32.96 | 37.16 | 41.58 | 58.90 |
| Group | 86.05 +/− | 116.20 +/− | 196.48 +/− | 276.41 +/− | 450.58 +/− | 598.84 +/− | 1239.53 +/− | 1521.86 +/− |
| 02 | 1.67 | 2.58 | 6.67 | 10.31 | 35.61 | 48.72 | 48.49 | 64.89 |
| Group | 86.36 +/− | 98.82 +/− | 125.97 +/− | 232.26 +/− | 354.70 +/− | 507.62 +/− | 1053.57 +/− | 1151.02 +/− |
| 03 | 1.91 | 2.80 | 5.40 | 8.96 | 20.11 | 39.85 | 26.03 | 28.62 |
| Group | 86.71 +/− | 94.50 +/− | 111.80 +/− | 214.76 +/− | 315.64 +/− | 315.55 +/− | 748.19 +/− | 875.67 +/− |
| 04 | 1.98 | 1.32 | 3.79 | 8.58 | 23.29 | 21.41 | 30.86 | 33.88 |
| Group | 86.32 +/− | 97.27 +/− | 129.15 +/− | 212.99 +/− | 334.58 +/− | 345.20 +/− | 680.16 +/− | 830.27 +/− |
| 05 | 1.87 | 2.27 | 6.80 | 9.88 | 23.59 | 36.54 | 30.94 | 65.56 |

As shown in Table 2, surprisingly, similar reduction of tumor volume was found in both Groups 04 and 05 even though the mice in group 05 did not receive 2-[3-[(3R)-3-[[2-chloro-3-(trifluoromethyl)phenyl]methyl-(2,2-diphenylethyl)amino]butoxy]phenyl]acetic acid for two days out of every seven.

### Other Embodiments

All literature and similar material cited in this application, including, but not limited to, patents, patent applications, articles, books, treatises, and web pages, regardless of the format of such literature and similar materials, are expressly incorporated by reference in their entirety. In the event that one or more of the incorporated literature and similar materials differs from or contradicts this application, including but not limited to defined terms, term usage, described techniques, or the like, this application controls.

While the methods have been described in conjunction with various embodiments and examples, it is not intended that the methods be limited to such embodiments or examples. On the contrary, the present disclosure encompasses various alternatives, modifications, and equivalents, as will be appreciated by those of skill in the art.

While the methods have been particularly shown and described with reference to specific illustrative embodiments, it should be understood that various changes in form and detail may be made without departing from the spirit and scope of the present disclosure. Therefore, all embodiments that come within the scope and spirit of the present disclosure, and equivalents thereto, are intended to be claimed. The claims, descriptions and diagrams of the methods, systems, and assays of the present disclosure should not be read as limited to the described order of elements unless stated to that effect.

What is claimed is:

1. A method of increasing the level of ABCA1, ABCG1, ABCG5, ABCG8, SREBP1, ApoE, and/or cholesteryl ester transfer protein mRNA in a subject, the method comprising administering to the subject an effective amount of 2-[3-[(3R)-3-[[2-chloro-3-(trifluoromethyl)phenyl]methyl-(2,2-diphenylethyl)amino]butoxy]phenyl]acetic acid, or a pharmaceutically acceptable salt thereof, at least once daily for four to six days followed by one to three days without administration of 2-[3-[(3R)-3-[[2-chloro-3-(trifluoromethyl)phenyl]methyl-(2,2-diphenylethyl)amino]butoxy]phenyl]acetic acid, or a pharmaceutically acceptable salt thereof.

2. A method of decreasing the level of myeloid derived suppressor cells (MDSCs) in a subject, the method comprising administering to the subject an effective amount of 2-[3-[(3R)-3-[[2-chloro-3-(trifluoromethyl)phenyl]methyl-(2,2-diphenylethyl)amino]butoxy]phenyl]acetic acid, or a pharmaceutically acceptable salt thereof, at least once daily for four to six days followed by one to three days without administration of 2-[3-[(3R)-3-[[2-chloro-3-(trifluoromethyl)phenyl]methyl-(2,2-diphenylethyl)amino]butoxy]phenyl]acetic acid, or a pharmaceutically acceptable salt thereof.

3. A method of increasing the level of activated T-cells in a subject, the method comprising administering to the subject an effective amount of 2-[3-[(3R)-3-[[2-chloro-3-(trifluoromethyl)phenyl]methyl-(2,2-diphenylethyl)amino]butoxy]phenyl]acetic acid, or a pharmaceutically acceptable salt thereof, at least once daily for four to six days followed by one to three days without administration of 2-[3-[(3R)-3-[[2-chloro-3-(trifluoromethyl)phenyl]methyl-(2,2-diphenylethyl)amino]butoxy]phenyl]acetic acid, or a pharmaceutically acceptable salt thereof.

4. The method of claim 3, wherein the activated T-cells are PD1+, GITR+, or Lag3+CD8 T-cells.

5. A method of increasing ApoE levels in a subject, the method comprising administering to the subject an effective amount of 2-[3-[(3R)-3-[[2-chloro-3-(trifluoromethyl)phenyl]methyl-(2,2-diphenylethyl)amino]butoxy]phenyl]acetic acid, or a pharmaceutically acceptable salt thereof, at least once daily for four to six days followed by one to three days without administration of 2-[3-[(3R)-3-[[2-chloro-3-(trifluoromethyl)phenyl]methyl-(2,2-diphenylethyl)amino]butoxy]phenyl]acetic acid, or a pharmaceutically acceptable salt thereof.

6. A method of treating ApoE-related cancer in a subject in need thereof, the method comprising administering to the subject an effective amount of 2-[3-[(3R)-3-[[2-chloro-3-(trifluoromethyl)phenyl]methyl-(2,2-diphenylethyl)amino]butoxy]phenyl]acetic acid, or a pharmaceutically acceptable salt thereof, at least once daily for four to six days followed by one to three days without administration of 2-[3-[(3R)-3-[[2-chloro-3-(trifluoromethyl)phenyl]methyl-(2,2-diphenylethyl)amino]butoxy]phenyl]acetic acid, or a pharmaceutically acceptable salt thereof.

7. The method of any one of claims 1 to 6, wherein the method comprises administering to the subject an effective amount of 2-[3-[(3R)-3-[[2-chloro-3-(trifluoromethyl)phenyl]methyl-(2,2-diphenylethyl)amino]butoxy]phenyl]acetic acid, or a pharmaceutically acceptable salt thereof, at least once daily for five days followed by two days without administration of 2-[3-[(3R)-3-[[2-chloro-3-(trifluoromethyl)phenyl]methyl-(2,2-diphenylethyl)amino]butoxy]phenyl]acetic acid, or a pharmaceutically acceptable salt thereof.

8. The method of any one of claims 1 to 7, wherein the method comprises administering the effective amount of 2-[3-[(3R)-3-[[2-chloro-3-(trifluoromethyl)phenyl]methyl-(2,2-diphenylethyl)amino]butoxy]phenyl]acetic acid, or a pharmaceutically acceptable salt thereof, twice daily for five days followed by two days without administration of 2-[3-[(3R)-3-[[2-chloro-3-(trifluoromethyl)phenyl]methyl-(2,2-diphenylethyl)amino]butoxy]phenyl]acetic acid, or a pharmaceutically acceptable salt thereof.

9. The method of any one of claims 6 to 8, wherein the ApoE cancer is breast cancer, colon cancer, renal cell cancer, lung cancer, hepatocellular carcinoma, gastric cancer, ovarian cancer, pancreatic cancer, esophageal cancer, prostate cancer, sarcoma, bladder cancer, neuroendocrine cancer, lymphoma, squamous cell carcinoma of the head and neck, or melanoma.

10. The method of claim 9, wherein the lung cancer is non-small cell lung cancer or small-cell lung cancer.

11. The method of any one of claims 1 to 10, wherein the effective amount of 2-[3-[(3R)-3-[[2-chloro-3-(trifluoromethyl)phenyl]methyl-(2,2-diphenylethyl)amino]butoxy]phenyl]acetic acid, or a pharmaceutically acceptable salt thereof is about 80 mg to about 160 mg per administration.

12. The method of any one of claims 1 to 11, wherein the method comprises administering about 80 mg of 2-[3-[(3R)-3-[[2-chloro-3-(trifluoromethyl)phenyl]methyl-(2,2-diphenylethyl)amino]butoxy]phenyl]acetic acid, or a pharmaceutically acceptable salt thereof, twice daily for five days followed by two days without administration of 2-[3-[(3R)-3-[[2-chloro-3-(trifluoromethyl)phenyl]methyl-(2,2-diphenylethyl)amino]butoxy]phenyl]acetic acid, or a pharmaceutically acceptable salt thereof.

13. The method of any one of claims 1 to 12, wherein the method further comprises administering an additional anti-cancer therapy to the subject.

14. The method of claim 13, wherein the additional anti-cancer therapy comprises surgery, radiation, chemotherapy, and/or immunotherapy.

15. The method of claim 14, wherein the additional anti-cancer therapy comprises chemotherapy.

16. The method of claim 15, wherein the chemotherapy comprises docetaxel.

17. The method of claim 16, wherein the method comprises administering an effective amount of docetaxel to the subject once every seven days.

18. The method of claim 17, wherein the effective amount of docetaxel is at least 28 mg/m².

19. The method of claim 18, wherein the effective amount of docetaxel is about 28 mg/m² to about 35 mg/m².

20. The method of claim 14, wherein the additional anti-cancer therapy comprises chemotherapy and immunotherapy.

21. The method of claim 20, wherein the anti-cancer therapy comprises carboplatin or cisplatin, pemetrexed, and pembrolizumab.

22. The method of claim 21, wherein the method comprises administering to the subject an effective amount of pembrolizumab once every twenty-one days.

23. The method of claim 22, wherein the effective amount of pembrolizumab is about 200 mg.

24. The method of any one of claims 20 to 23, wherein the method comprises administering to the subject an effective amount of carboplatin or cisplatin once every twenty-one days.

25. The method of claim 24, wherein the effective amount of carboplatin or cisplatin is calculated using the formula:
Total dose (mg)=(Target area under the curve)×(subject's glomerular filtration rate+25)
wherein the target area under the curve is 4 mg/mL*min to 6 mg/mL*min and the subject's glomerular filtration rate was measured by Cr-EDTA clearance.

26. The method of claim 24, wherein the effective amount of carboplatin or cisplatin is about 300 mg/m² to about 360 mg/m².

27. The method of any one of claims 20 to 26, wherein the method comprises administering to the subject an effective amount of pemetrexed once every twenty-one days.

28. The method of claim 27, wherein the effective amount of pemetrexed is 500 mg/m².

29. The method of any one of claims 20 to 28, wherein the method further comprises administering to the subject an effective amount of folic acid, vitamin B12, and/or corticosteroids.

30. The method of claim 29, wherein the method comprises administering to the subject an effective amount of corticosteroids twice per day for three days prior to administration of pemetrexed.

31. The method of any one of claims 1 to 30, wherein the method further comprises administering to the subject an effective amount of a statin.

32. The method of claim 31, wherein the statin is rosuvastatin or atorvastatin.

33. The method of any one of claims 1 to 32, wherein the method further comprises administering to the subject an effective amount of an anti-emetic agent, an anti-diarrheal agent, an appetite stimulant, a general stimulant, a bisphosphonate, a gonadotrophin releasing hormone agonist, growth factors, and/or an LHRH agonist.

34. The method of any one of claims 1 to 33, wherein the cancer is lung cancer.

35. The method of claim 34, wherein the lung cancer is small cell lung cancer.

36. The method of claim 34, wherein the lung cancer is non-small cell lung cancer.

37. The method of claim 36, wherein the non-small cell lung cancer is a non-squamous cell carcinoma.

38. The method of any one of claims 1 to 33, wherein the cancer is a neuroendocrine tumor.

39. The method of any one of claims 1 to 38, wherein the cancer is resistant to platinum-containing chemotherapy, a PD-1 inhibitor, a PD-L1 inhibitor, a CTLA-4 inhibitor, an antimitotic agent, a topoisomerase inhibitor, an antimetabolite, an angiogenesis inhibitor, a kinase inhibitor, and/or an alkylating agent.

40. The method of any one of claims 1 to 38, wherein the cancer progressed on or after treatment with platinum-containing chemotherapy, a PD-1 inhibitor, a PD-L1 inhibitor, an angiogenesis inhibitor, a kinase inhibitor, and/or an alkylating agent.

41. The method of any one of claims 1 to 38, wherein the cancer has been determined to be, or is predicted to be, resistant to a PD-1 inhibitor, a PD-L1 inhibitor, a CTLA-4 inhibitor, a topoisomerase inhibitor, an antimetabolite, an angiogenesis inhibitor, a kinase inhibitor, and/or an alkylating agent.

42. The method of any one of claims 1 to 41, the cancer has a PDL-1 expression level of less than 1% when tested in an immunohistochemistry assay.

43. The method of any one of claims 1 to 42, wherein the cancer is metastatic and/or locally advanced.

44. The method of any one of claims 1 to 43, wherein the cancer is unresectable.

45. The method of any one of claims 1 to 44, wherein the risk of adverse events is reduced in comparison to administration of the effective amount of 2-[3-[(3R)-3-[[2-chloro-3-(trifluoromethyl)phenyl]methyl-(2,2-diphenylethyl)amino]butoxy]phenyl]acetic acid, or a pharmaceutically acceptable salt thereof, at least once daily for seven consecutive days.

46. The method of claim 45, wherein the risk of neutropenia is reduced in comparison to administration of the effective amount of 2-[3-[(3R)-3-[[2-chloro-3-(trifluoromethyl)phenyl]methyl-(2,2-diphenylethyl)amino]butoxy]phenyl]acetic acid, or a pharmaceutically acceptable salt thereof, at least once daily for seven consecutive days.

47. The method of claim 45 or 46, wherein the risk of immune-related adverse events, hypertriglyceridemia and/or hypercholesterolemia is reduced in comparison to administration of the effective amount of 2-[3-[(3R)-3-[[2-chloro-3-(trifluoromethyl)phenyl]methyl-(2,2-diphenylethyl)amino]butoxy]phenyl]acetic acid, or a pharmaceutically acceptable salt thereof, at least once daily for seven consecutive days.

48. The method of any one of claims 1 to 47, wherein the level of neutrophils in a sample from the subject are increased in comparison to a mean of the level of neutrophils in a plurality of subjects administered the effective amount of 2-[3-[(3R)-3-[[2-chloro-3-(trifluoromethyl)phenyl]methyl-(2,2-diphenylethyl)amino]butoxy]phenyl]acetic acid, or a pharmaceutically acceptable salt thereof, at least once daily for seven consecutive days.

49. The method of any one of claims 1 to 48, wherein the level of triglycerides and/or cholesterol in a sample from the subject are decreased in comparison to a mean of the level of triglycerides and/or cholesterol in a plurality of subjects administered the effective amount of 2-[3-[(3R)-3-[[2-chloro-3-(trifluoromethyl)phenyl]methyl-(2,2-diphenylethyl)amino]butoxy]phenyl]acetic acid, or a pharmaceutically acceptable salt thereof, at least once daily for seven consecutive days.

50. The method of any one of claims 1 to 49, wherein the 2-[3-[(3R)-3-[[2-chloro-3-(trifluoromethyl)phenyl]methyl-(2,2-diphenylethyl)amino]butoxy]phenyl]acetic acid is administered orally.

51. The method of any one of claims 1 to 50, wherein the 2-[3-[(3R)-3-[[2-chloro-3-(trifluoromethyl)phenyl]methyl-(2,2-diphenylethyl)amino]butoxy]phenyl]acetic acid is administered once or twice per day.

52. The method of any one of claims 1 to 51, wherein about 80 mg of 2-[3-[(3R)-3-[[2-chloro-3-(trifluoromethyl)phenyl]methyl-(2,2-diphenylethyl)amino]butoxy]phenyl]acetic acid is administered in each dose.

53. The method of any one of claims 1 to 52, wherein the method comprises at least 21 days of treatment.

54. The method of any one of claims 1 to 53, wherein the method comprises at least 28 days of treatment.

55. A method of treating ApoE-related cancer in a subject in need thereof, the method comprising administering to the subject about 80 mg of 2-[3-[(3R)-3-[[2-chloro-3-(trifluoromethyl)phenyl]methyl-(2,2-diphenylethyl)amino]butoxy]phenyl]acetic acid, or a pharmaceutically acceptable salt thereof, wherein for each seven day period of treatment, the 2-[3-[(3R)-3-[[2-chloro-3-(trifluoromethyl)phenyl]methyl-(2,2-diphenylethyl)amino]butoxy]phenyl]acetic acid, or a pharmaceutically acceptable salt thereof is administered twice daily for five consecutive days followed by two consecutive days without administration of 2-[3-[(3R)-3-[[2-chloro-3-(trifluoromethyl)phenyl]methyl-(2,2-diphenylethyl)amino]butoxy]phenyl]acetic acid, or a pharmaceutically acceptable salt thereof.

56. The method of claim 55, wherein the ApoE cancer is breast cancer, colon cancer, renal cell cancer, lung cancer, hepatocellular carcinoma, gastric cancer, ovarian cancer, pancreatic cancer, esophageal cancer, prostate cancer, sarcoma, bladder cancer, neuroendocrine cancer, lymphoma, squamous cell carcinoma of the head and neck, or melanoma.
